(19) United States
(12) Patent Application Publication
(10) Pub. No.: US 2026/0267056 A1
(43) Pub. Date: Sep. 10, 2026

(54) COMPOUND, COMPOSITION, CURED PRODUCT, OPTICALLY ANISOTROPIC BODY, OPTICAL ELEMENT, AND LIGHT GUIDING ELEMENT

(71) Applicant: FUJIFILM Corporation, Tokyo (JP)

(72) Inventors: Keisuke NAKANISHI, Minamiashigara-shi (JP); Keisuke KODAMA, Minamiashigara-shi (JP); Yuki FUKUSHIMA, Minamiashigara-shi (JP)

(73) Assignee: FUJIFILM Corporation, Tokyo (JP)

(21) Appl. No.: 19/466,567

(22) Filed: Feb. 2, 2026

Related U.S. Application Data
(63) Continuation of application No. PCT/JP2024/028907, filed on Aug. 13, 2024.

(30) Foreign Application Priority Data
Aug. 18, 2023 (JP) 2023-133395

(51) Int. Cl. F21V 8/00; G02B 5/30; G03F 7/031

(52) U.S. Cl. CPC G02B 6/0065; G02B 5/3016; G03F 7/031

(57) ABSTRACT

An object of the present invention is to provide a compound that can form an optically anisotropic layer having a high refractive index ne for extraordinary rays and a high birefringence index Δn in the optically anisotropic layer after curing and that has excellent pattern alignment properties. In addition, another object of the present invention is to provide a composition, a cured product, an optically anisotropic body, an optical element, and a light guide element.

### CROSS-REFERENCE TO RELATED APPLICATIONS

This application is a Continuation of PCT International Application No. PCT/JP2024/028907 filed on Aug. 13, 2024, which claims priority under 35 U.S.C. § 119 (a) to Japanese Patent Application No. 2023-133395 filed on Aug. 18, 2023. The above applications are hereby expressly incorporated by reference, in their entirety, into the present application.

### BACKGROUND OF THE INVENTION

### 1. Field of the Invention

The present invention relates to a compound, a composition, a cured product, an optically anisotropic body, an optical element, and a light guide element.

### 2. Description of the Related Art

In recent years, since polarized light has been used in various optical devices or systems, there is a demand for an optical element for controlling reflection, focusing, divergence, and the like of polarized light. In particular, to reduce a variation in performance of the optical element with respect to an incidence angle of light incident on the optical element, an optical element having a large refractive index ne for extraordinary rays is required.

As a compound that provides a high refractive index ne for extraordinary rays, for example, a compound including a tolan structure moiety (hereinafter, also referred to as a “tolan compound”) is known. Since the tolan compound has a relatively high refractive index, an optically anisotropic body formed of a liquid crystal composition including the tolan compound has a high refractive index and tends to exhibit low angle-of-incidence dependence of diffraction efficiency, even in a case where the tolan compound itself has liquid crystallinity or in a case where the tolan compound itself does not have liquid crystallinity and is used in combination with another liquid crystal compound.

For example, WO2019/182129A discloses a liquid crystal compound having a tolan structure moiety.

### SUMMARY OF THE INVENTION

As a result of studying the liquid crystal compound described in WO2019/182129A, the present inventors have found that the refractive index ne for extraordinary rays after curing the optically anisotropic layer is insufficient in the optically anisotropic layer produced using the liquid crystal compound, and have clarified that improvement is required.

In addition, the optically anisotropic layer after curing is required to have a high birefringence index Δn, and is required to have excellent pattern alignment properties in which the compound is aligned along a pattern.

Therefore, an object of the present invention is to provide a compound that can form an optically anisotropic layer having a high refractive index ne for extraordinary rays and a high birefringence index Δn in the optically anisotropic layer after curing and that has excellent pattern alignment properties.

In addition, another object of the present invention is to provide a composition, a cured product, an optically anisotropic body, an optical element, and a light guide element.

In order to achieve the above objects, the inventors of the present invention carried out intensive examinations. As a result, the inventors have found that the objects can be achieved by the following constitution.

[1] A compound represented by Formula (I).

[2] The compound according to [1], in which, in Formula (Ia), two of X¹, X², X³, and X⁴ represent —C(*)—, and the other two of X¹, X², X³, and X⁴ represent —C(R^{A})=.

[3] The compound according to [1] or [2], in which, in Formula (Ia), X¹ and X⁴ represent —C(*)═.

[4] The compound according to any one of [1] to [3], in which, in Formula (I), at least one of n1 pieces of Z¹'s is —C≡C—.

[5] The compound according to any one of [1] to [4], in which, in Formula (I), at least two selected from among n1 pieces of A¹ and one piece of A² are each the fused-ring structure represented by Formula (Ia).

[6] The compound according to any one of [1] to [5], in which, in Formula (I), at least two selected from among n1 pieces of A¹ and one piece of A² are each the fused-ring structure represented by Formula (Ia), and X¹ and X⁴ in the fused-ring structure represented by Formula (Ia) represent —C(*)═.

[7] The compound according to any one of [1] to [6], in which, in Formula (I), at least one of P¹ or P² represents a polymerizable group selected from the group consisting of Formula (P-1) to Formula (P-19).

[8] The compound according to [7], in which, in Formula (I), the at least one of P¹ or P² represents a polymerizable group selected from Formula (P-1) or Formula (P-2).

[9] The compound according to any one of [1] to [8], in which, in Formula (I), n1 represents an integer of 3 to 7.

[10] A composition comprising the compound according to any one of [1] to [9].

[11] The composition according to [10], further comprising a polymerization initiator.

[12] The composition according to or [11], further comprising a chiral agent.

[13] The composition according to any one of to [12], in which the composition has liquid crystallinity.

[14] A cured product obtained by curing the composition according to any one of to [13].

[15] An optically anisotropic body obtained by curing the composition according to any one of to [13].

[16] The optically anisotropic body according to [15], in which the optically anisotropic body has an alignment pattern in which an orientation of an optical axis derived from the compound continuously rotates in at least one in-plane direction.

[17] An optical element comprising the optically anisotropic body according to [16].

[18] A light guide element comprising: the optical element according to [17]; and a light guide plate.

According to the present invention, it is possible to provide a compound that can form an optically anisotropic layer having a high refractive index ne for extraordinary rays and a high birefringence index Δn in the optically anisotropic layer after curing and that has excellent pattern alignment properties.

In addition, according to the present invention, it is also possible to provide a composition, a cured product, an optically anisotropic body, an optical element, and a light guide element.

### BRIEF DESCRIPTION OF THE DRAWINGS

FIG. 1 is a schematic view showing an embodiment of an optically anisotropic layer.

FIG. 2 is a schematic plan view of the optically anisotropic layer shown in FIG. 1.

FIG. 3 is a conceptual diagram showing an action of the optically anisotropic layer shown in FIG. 2.

FIG. 4 is a conceptual diagram showing an action of the optically anisotropic layer shown in FIG. 2.

FIG. 5 is a schematic view showing another example of the optically anisotropic layer.

FIG. 6 is a schematic view showing another example of the optically anisotropic layer.

### DESCRIPTION OF THE PREFERRED EMBODIMENTS

Hereinafter, the present invention will be described in detail.

Although the configuration requirements to be described below may be described based on representative embodiments of the present invention, the present invention is not limited to such embodiments.

In each of the drawings, for easy visual recognition, the reduced scale of components is different from the actual scale.

In addition, in this specification, a numerical range represented using “to” means a range that includes numerical values written before and after “to” as a lower limit and an upper limit.

In addition, in the present specification, “perpendicular” or “parallel” regarding an angle represents a range of the exact angle #10°.

In addition, in the present specification, n represents a refractive index, ne represents a refractive index in a slow axis direction, and no represents a refractive index in a fast axis direction. Unless otherwise specified, n, ne, and no mean values at a wavelength of 550 nm.

In addition, in the present specification, n, ne, and no are values measured by ellipsometry.

In addition, in the present specification, “(meth)acryloyloxy group” is a notation representing both an acryloyloxy group and a methacryloyloxy group, and “(meth)acrylate” is a notation representing both an acrylate and a methacrylate.

In addition, in a notation for a group (atomic group) in the present specification, in a case where the group is denoted without specifying whether it is substituted or unsubstituted, the group includes both a group having no substituent and a group having a substituent. For example, an “alkyl group” includes not only an alkyl group having no substituent (unsubstituted alkyl group), but also an alkyl group having a substituent (substituted alkyl group).

In addition, the bonding direction of a divalent group described in the present specification is not limited unless otherwise specified. For example, in a case where Y in a compound represented by Formula “X—Y—Z” is —COO—, Y may be —CO—O— or —O—CO—. In addition, the above-described compound may be “X—CO—O—Z” or “X—O—CO—Z”.

In addition, in the present specification, in a case where a “substituent” is simply described, examples of the substituent include the following substituent L. In addition, in the present specification, in a case where a “substituent L” is described, the substituent L is intended to be the following substituent L.

### (Substituent L)

Examples of the substituent L include an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amido group, a cyano group, a nitro group, a halogen atom, an NCS group, an SO₂NCS group, an OCN group, a trifluoromethyl group, a halogen, a polymerizable group, and the like. Provided that, in a case where the group described as the substituent L contains —CH₂— (methylene group), a group in which at least one —CH₂— contained in the group is substituted with —O—, —CO—, —CH—CH—, or —C≡C— is also included in the substituent L. For example, in a case where the above-described group has two or more —CH₂-'s, one —CH₂— may be substituted with —O— and one —CH₂— adjacent to the —O— may be substituted with —CO— to form an ester group (—O—CO—). Here, in a case where the group described as the substituent L has a hydrogen atom, a group in which at least one hydrogen atom—in the group is substituted with at least one selected from the group consisting of a fluorine atom and a polymerizable group is also included in the substituent L. In addition, in a case where at least one —CH₂— is substituted with —O—, —CO—, —CH═CH—, or —C≡C—, the number of carbon atoms in the state after the substitution satisfies the predetermined numerical range. That is, for example, in a case where an alkyl group having 1 to 10 carbon atoms is taken as an example, in a case where at least one —CH₂— of the alkyl group is substituted with —O—, —CO—, —CH—CH—, or —C≡C—, the number of carbon atoms of the alkyl group after the substitution satisfies 1 to 10.

Furthermore, examples of the above-described polymerizable group include an ethylenically unsaturated group, a ring-polymerizable group, and the like, and among these, a substituent selected from a polymerizable group P, which will be described later, is preferable.

Among these, the substituent L is preferably an alkyl group having 1 to 10 carbon atoms, an alkoxy group having 1 to 10 carbon atoms, an alkanoyl group having 1 to 10 carbon atoms, an alkanoyloxy group having 1 to 10 carbon atoms, an alkyloxycarbonyl group having 2 to 10 carbon atoms, a trifluoromethyl group, a hydroxy group, a carboxy group, a cyano group, a nitro group, or a halogen atom, more preferably an alkyl group having 1 to 10 carbon atoms, an alkoxy group having 1 to 10 carbon atoms, an alkanoyl group having 2 to 10 carbon atoms, an alkanoyloxy group having 2 to 10 carbon atoms, an alkyloxycarbonyl group having 2 to 10 carbon atoms, a trifluoromethyl group, or a halogen atom, and still more preferably an alkyl group having 1 to 6 carbon atoms, an alkoxy group having 1 to 6 carbon atoms, an alkanoyl group having 2 to 6 carbon atoms, an alkanoyloxy group having 2 to 6 carbon atoms, an alkyloxycarbonyl group having 2 to 6 carbon atoms, a trifluoromethyl group, or a fluorine atom.

In addition, in the present specification, in a case of simply referring to a “polymerizable group”, examples of the polymerizable group include a polymerizable group capable of addition polymerization and ring polymerization, for examples, the following polymerizable group P.

### (Polymerizable Group P)

Examples of the polymerizable group P include a group represented by any one of Formula (P-1) to Formula (P-19) below. In the following formulae, * represents a bonding position, Me represents a methyl group, and Et represents an ethyl group. Among these, Formula (P-1) or Formula (P-2) (a (meth)acryloyloxy group) is preferable.

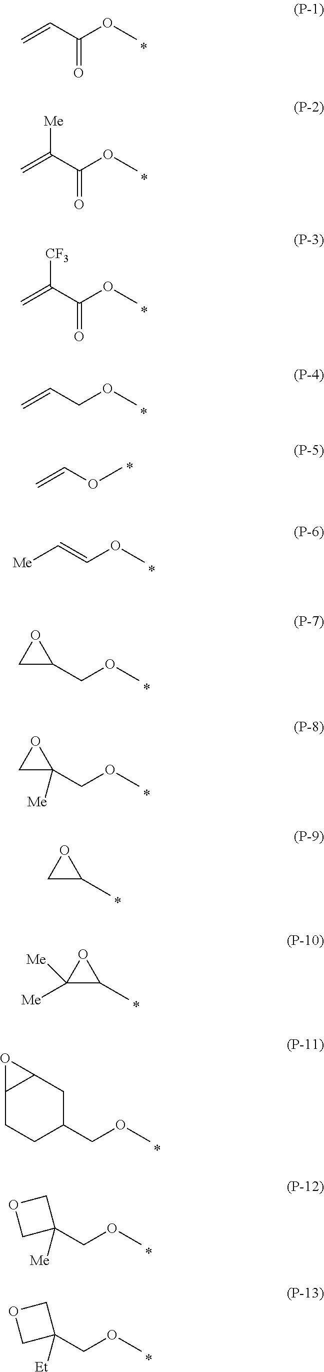

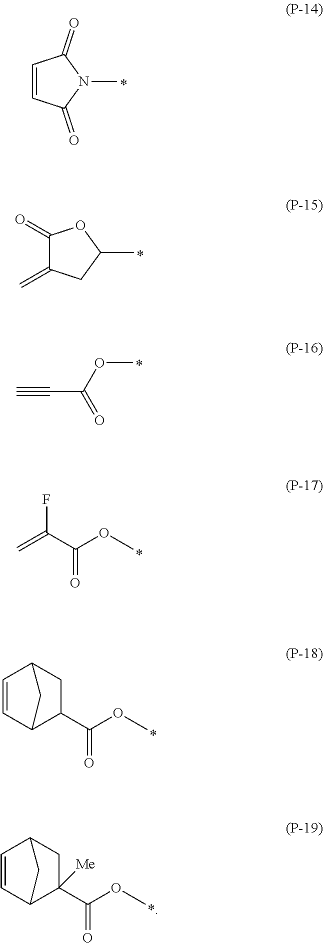

In addition, in the present specification, a “solid content” of a composition refers to components which form an optically anisotropic layer formed of the composition, and in a case where the composition includes a solvent (an organic solvent, water, and the like), the solid content means all components except the solvent. In addition, in a case where the components are components which form an optically anisotropic layer, the components are considered to be solid contents even in a case where the components are liquid components.

In addition, in the present specification, unless otherwise specified, a thickness of a layer is an average value of the thicknesses obtained by observing a cross section cut by a microtome with a scanning electron microscope (SEM) or a transmission electron microscope (TEM) and measuring the thickness at 10 points.

### [Compound Represented by Formula (I)]

A feature point of the compound represented by Formula (I) (hereinafter, also referred to as a “specific compound”) includes having a polymerizable group and a fused-ring structure represented by Formula (Ia) described later.

The optically anisotropic layer obtained by curing the composition containing the specific compound has a high refractive index ne for extraordinary rays and a high birefringence index Δn. In addition, the specific compound has excellent pattern aligning properties. The action mechanism is not always clear, but the present inventors estimate as follows.

The fused-ring structure represented by Formula (Ia) described later includes a sulfur atom having a large atomic refraction. In addition, the polymerizable group causes cure shrinkage during polymerization, which improves the density of the film and further improves the refractive index.

Furthermore, the refractive index of the liquid crystal compound is classified into a refractive index ne in a compound major axis direction and a refractive index no in a compound minor axis direction, and usually, the relationship of ne>no is satisfied. In the specific optical system, from the purpose of maximizing the refractive index for polarization, it is required to particularly increase the refractive index ne of the compound major axis. It is presumed that, since the compound has the fused-ring structure represented by Formula (Ia), the liquid crystallinity and the alignment degree of the compound are improved and ne is particularly increased.

In addition, the present inventors have confirmed that, since the alignment degree is decreased by polymerization, ne may be decreased by polymerization depending on the structure of the compound. However, in the specific compound, it is presumed that, since the intermolecular force of the fused-ring structure represented by Formula (Ia) described later is high, the film densification by polymerization readily proceeds and ne is easily increased.

In addition, it has been confirmed that the optically anisotropic layer obtained by curing the composition containing the specific compound has a high birefringence index Δn, and the specific compound has excellent pattern aligning properties.

Hereinafter, the fact that the refractive index ne of the optically anisotropic layer obtained by curing the composition containing the specific compound for extraordinary rays is higher, the birefringence index Δn of the optically anisotropic layer obtained by curing the composition containing the specific compound is higher, and/or the pattern aligning properties of the specific compound are more excellent is also referred to as “the effect of the present invention is more excellent”.

Hereinafter, the compound represented by Formula (I) (specific compound) will be described in detail.

### [Compound Represented by Formula (I)]

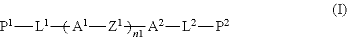

In Formula (I), P¹ and P² each independently represent a hydrogen atom, a polymerizable group, or a monovalent substituent, where at least one of P¹ or P² is a polymerizable group. From the viewpoint that the effect of the present invention is more excellent, it is preferable that both P¹ and P² are polymerizable groups.

Examples of the polymerizable group represented by P¹ and P² include an ethylenically unsaturated group and a ring-polymerizable group, and specific examples thereof include a substituent selected from the above-described polymerizable group P.

Examples of the monovalent substituent represented by P¹ and P² include the substituent L. The substituent L is as described above, and is preferably an alkyl group, an alkoxy group, a cyano group, or an NCS group, and more preferably an NCS group.

L¹ and L² each independently represent a single bond or an alkylene group having 20 or less carbon atoms, where any —CH₂— of the alkylene group may be substituted with —O—, —S—, —NR—, —CO—, or —CS—, any —(CH₂)₂— of the alkylene group may be substituted with —CH═CH— or —C≡C—, and any hydrogen atom of the alkylene group may be substituted with a fluorine atom or a chlorine atom. It is noted that in the alkylene group, in a case where any —CH₂— and —(CH₂)₂— are substituted, the number of carbon atoms in the state after the substitution satisfies the predetermined numerical range described above.

The alkylene group having 20 or less carbon atoms may be linear, branched, or cyclic, but among these, is preferably linear or branched.

The number of carbon atoms in the alkylene group having 20 or less carbon atoms is preferably 1 to 15, more preferably 1 to 10, and still more preferably 1 to 4.

The above-described R represents a hydrogen atom or an alkyl group having 1 to 10 carbon atoms.

The alkyl group may be linear, branched, or cyclic, but is preferably linear or branched, and more preferably linear.

The number of carbon atoms in the alkyl group represented by R is preferably 1 to 6 and more preferably 1 to 3.

In a case where a plurality of R's are present in the formula, the plurality of R's may be the same as or different from each other.

The R is preferably a hydrogen atom or an alkyl group having 1 to 3 carbon atoms, and more preferably a hydrogen atom.

A¹ and A² each independently represent a divalent aromatic ring group which may have a substituent or a divalent alicyclic group which may have a substituent, where at least one selected from among n1 pieces of A¹ and one piece of A² in Formula (1) is a fused-ring structure represented by Formula (Ia).

Among these, from the viewpoint that the effect of the present invention is more excellent, it is preferable that two or more selected from among n1 pieces of A¹ and one piece of A² in Formula (I) are a fused-ring structure represented by Formula (Ia).

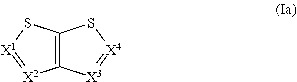

In Formula (Ia), two of X¹, X², X³, and X⁴ represent —C(*)═, and the other two of X¹, X², X³, and X⁴ each independently represent —N═ or —C(R^{A})—. R^{A} represents a hydrogen atom or a substituent. * represents a bonding position. That is, the fused-ring structure represented by Formula (Ia) corresponds to a divalent linking group having * as a bonding position.

In Formula (Ia), from the viewpoint that the effect of the present invention is more excellent, it is preferable that two of X¹, X², X³, and X⁴ represent —C(*)═, and the other two of X¹, X², X³, and X⁴ represent —C(R^{A})═.

Specific examples of the substituent represented by R^{A} include the substituent L described above.

Among those, R^{A} is preferably a hydrogen atom.

In Formula (Ia), from the viewpoint that the effect of the present invention is more excellent, it is preferable that X¹ and X⁴ represent —C(*)═ or X² and X³ represent —C(*)═, and it is more preferable that X¹ and X⁴ represent —C(*)—.

Examples of the divalent aromatic ring group represented by A¹ and A² include a divalent aromatic hydrocarbon ring group and a divalent aromatic heterocyclic group.

The aromatic hydrocarbon ring constituting the divalent aromatic hydrocarbon ring group may be monocyclic or polycyclic. In addition, the number of carbon atoms in the aromatic hydrocarbon ring group is preferably 6 to 20 and more preferably 6 to 10. Specific example of the aromatic hydrocarbon ring is preferably a benzene ring or a naphthalene ring, and more preferably a benzene ring.

The number of ring members in the aromatic heterocyclic ring constituting the divalent aromatic heterocyclic group is preferably 5 to 10 and more preferably 5 or 6. Examples of a heteroatom included in the aromatic heterocyclic ring include a nitrogen atom, an oxygen atom, and a sulfur atom. In addition, the number of carbon atoms in the aromatic heterocyclic ring is preferably 3 to 20 and more preferably 3 to 10. Specific examples of the aromatic hetero ring include a pyridine ring, a pyridazine ring, a pyrimidine ring, a pyrazine ring, a triazine ring, a thiophene ring, a thiazole ring, an imidazole ring, and the like.

The divalent aromatic ring group represented by A¹ and A² is preferably a divalent aromatic hydrocarbon ring group, and more preferably a divalent benzene ring group (phenylene group), a divalent thienothiophene ring group, a divalent thienothiazole ring group, a divalent thiazolothiazole ring group, a divalent benzothiophene ring group, or a divalent naphthalene ring group (naphthylene group).

Examples of the divalent alicyclic group represented by A¹ and A² include a divalent aliphatic hydrocarbon ring group and a divalent aliphatic heterocyclic group.

The aliphatic hydrocarbon ring constituting the divalent aliphatic hydrocarbon ring group may be monocyclic or polycyclic.

The number of ring members in the aliphatic heterocyclic ring preferably 3 to 20, more preferably 3 to 10, and still more preferably 5 or 6. Specific examples of the aliphatic hydrocarbon ring include a cyclopentane ring, a cyclohexane ring, a cycloheptane ring, a cyclooctane ring, a norbornene ring, and an adamantane ring. Among these, a cyclopentane ring or a cyclohexane ring is preferable.

The aliphatic heterocyclic ring constituting the divalent aliphatic heterocyclic group may be monocyclic or polycyclic.

Examples of a heteroatom included in the aliphatic heterocyclic ring include a nitrogen atom, an oxygen atom, and a sulfur atom. The number of ring members of the aliphatic heterocycle is not particularly limited, but is preferably 5 to 10. Specific examples of the aliphatic hetero ring include an oxolane ring, an oxane ring, a piperidine ring, a piperazine ring, and the like. In the aliphatic hetero ring, —CH₂-constituting the ring may be replaced with —CO—, and examples thereof include a phthalimide ring.

The divalent aromatic ring group and the divalent alicyclic group represented by A¹ and A² may have a substituent. Examples of the substituent include the substituent L described above. In addition, in a case where the divalent aromatic ring group and the divalent alicyclic group have a plurality of substituents (preferably, the substituents L), the substituents may form a ring by bonding to each other, to form a fused-ring structure.

Among these, from the viewpoint that the effect of the present invention is more excellent, A¹ and A² are preferably a divalent benzene ring group (phenylene group) which may have a substituent L, a divalent benzothiophene ring which may have a substituent L, or a divalent naphthalene ring group (naphthylene group) which may have a substituent L.

It is noted that in a case where a plurality of A¹'s are present in the formula, the plurality of A¹'s may be the same as or different from each other.

Z¹'s each independently represent a single bond, —O—, —S—, —CHRCHR—, —OCHR—, —CO—, —SO—, —SO₂—, —COO—, —CO—S—, —O—CO—O—, —CO—NR—, —SCHR—, —SO—CHR—, —SO₂—CHR—, —CF₂O—, —CF₂S—, —OCHRCHRO—, —SCHRCHRS—, —SO—CHRCHR—SO—, —SO₂—CHRCHR—SO₂—, —CH═CH—COO—, —CH═CH—OCO—, —CH—CH—CONR—, —CH═CH—COS—, —COO—CHRCHR—, —OCO—CHRCHR—, —COO—CHR—, —OCO—CHR—, —CR═CR—, —CR═N—, —N═CR—, —N═N—, —CR═N—N═CR—, —CF≡CF—, —C≡C—C≡C—, —C≡C—, or an alkylene group having 10 or less carbon atoms, where any —CH₂— of the alkylene group may be substituted with —O—, —S—, —NR—, —CO—, or —CS—, any —(CH₂)₂— of the alkylene group may be substituted with —CH═CH— or —C≡C—, and any hydrogen atom of the alkylene group may be substituted with a fluorine atom or a chlorine atom. It is noted that in the alkylene group, in a case where any —CH₂— and —(CH₂)₂— are substituted, the number of carbon atoms in the state after the substitution satisfies the predetermined numerical range described above.

The alkylene group having 10 or less carbon atoms may be linear, branched, or cyclic, but among these, is preferably linear or branched.

The number of carbon atoms in the alkylene group having 10 or less carbon atoms is preferably 1 to 6 and more preferably 1 to 3.

R represents a hydrogen atom or an alkyl group having 1 to 10 carbon atoms.

The alkyl group may be linear, branched, or cyclic, but is preferably linear or branched, and more preferably linear.

The number of carbon atoms in the above-described alkyl group represented by R is preferably 1 to 6 and more preferably 1 to 3.

It is noted that in the formula, in a case where a plurality of R's are present, the plurality of R's may be the same as or different from each other.

Among those, R is preferably a hydrogen atom.

Among these, Z¹ is preferably a single bond, —CHRCHR—, —OCHR—, —COO—, —CO—NH—, or —C≡C—, and more preferably —OCH₂— or —C≡C—.

It is noted that in a case where a plurality of Z¹'s are present in the formula, the plurality of Z¹'s may be the same as or different from each other. In Formula (I), it is preferable that at least one of Z¹'s is —C≡C—.

n represents an integer of 1 to 7.

From the viewpoint that the effect of the present invention is more excellent, n1 is preferably 1 to 4 and more preferably 2 or 3.

From the viewpoint that the pattern aligning properties of the specific compound are more excellent, n1 is also preferably 3 to 7.

In Formula (I), in a case where n1 is an integer of 2 or more, it is preferable that among the plurality of Z¹'s, two Z¹'s at positions adjacent to each other through A¹ are not both —C≡C—.

The case where n1 represents 3 will be described as an example. In a case where n1 represents 3, the structural moiety represented by -(A¹-Z¹)ₙ₁- in Formula (I) represents -A¹A-Z^{1a}-A^{1B}-Z^{1b}-A^{1C}-Z^{1c}-(provided that A¹-A to A¹C have the same meaning as A¹, and Z^{1a} to Z^{1c} have the same meaning as Z¹). In this case, it is preferable that Z^{1a} and Z^{1b} at positions adjacent to each other through A^{1B} are not simultaneously —C≡C—. That is, for example, in a case where Z^{1a} represents —C≡C—, it is preferable that Z^{1b} is a group (a single bond or a divalent linking group other than —C≡C-among the divalent linking groups represented by Z¹) that can be adopted by Z¹ other than —C≡C—. In addition, it is preferable that Z^{1b} and Z^{1c} at positions adjacent to each other through A^{1C} are also not simultaneously —C≡C—.

In Formula (I), in a case where n1 is an integer of 2 or more, in a case where among the plurality of Z¹'s, two Z¹'s at positions adjacent to each other through A¹ are both —C≡C—, it is preferable that A¹ in which two —C≡C-'s are linked is a structure represented by Formula (Ia). In the above-described aspect, it is preferable that Z¹ is bonded to X² and X³ in Formula (Ia).

Hereinafter, an example of the specific compound will be described, but the present invention is not limited thereto.

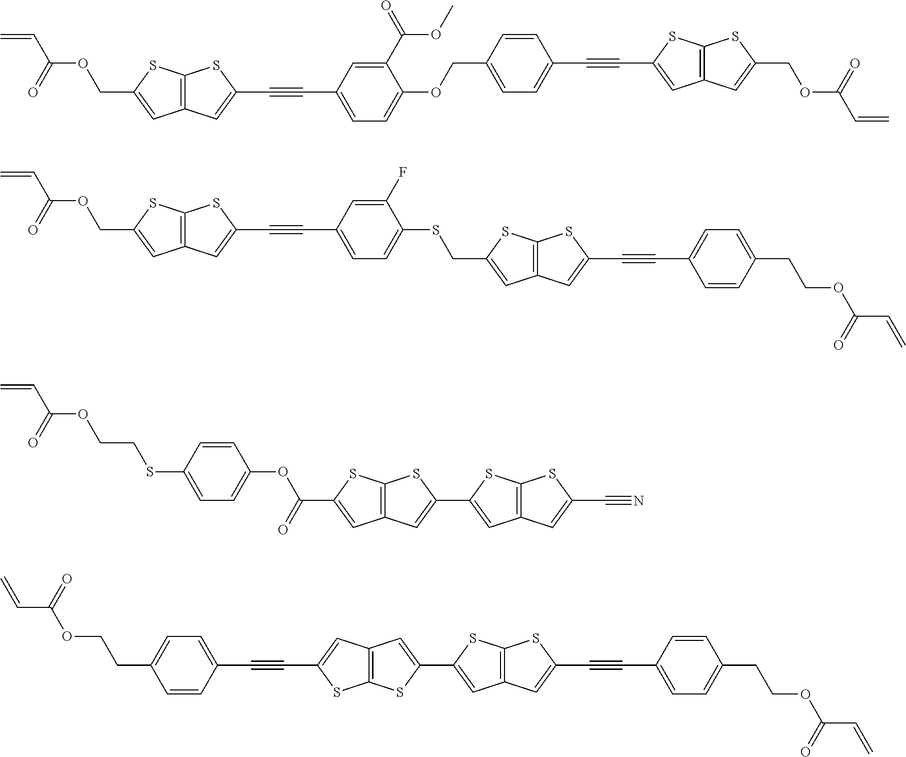

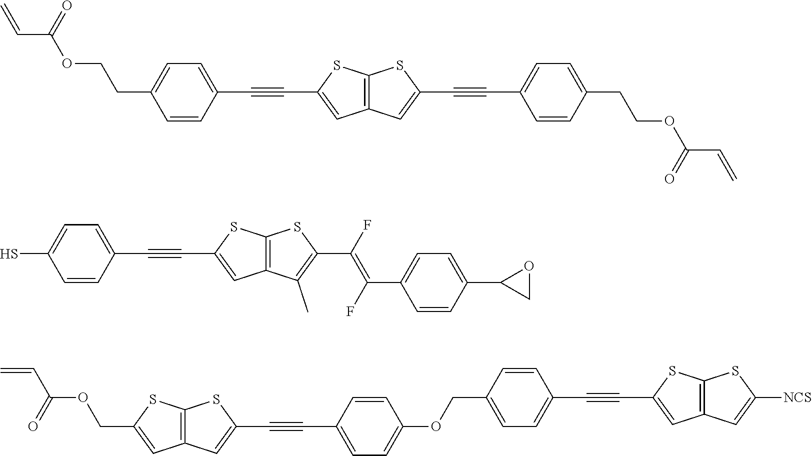

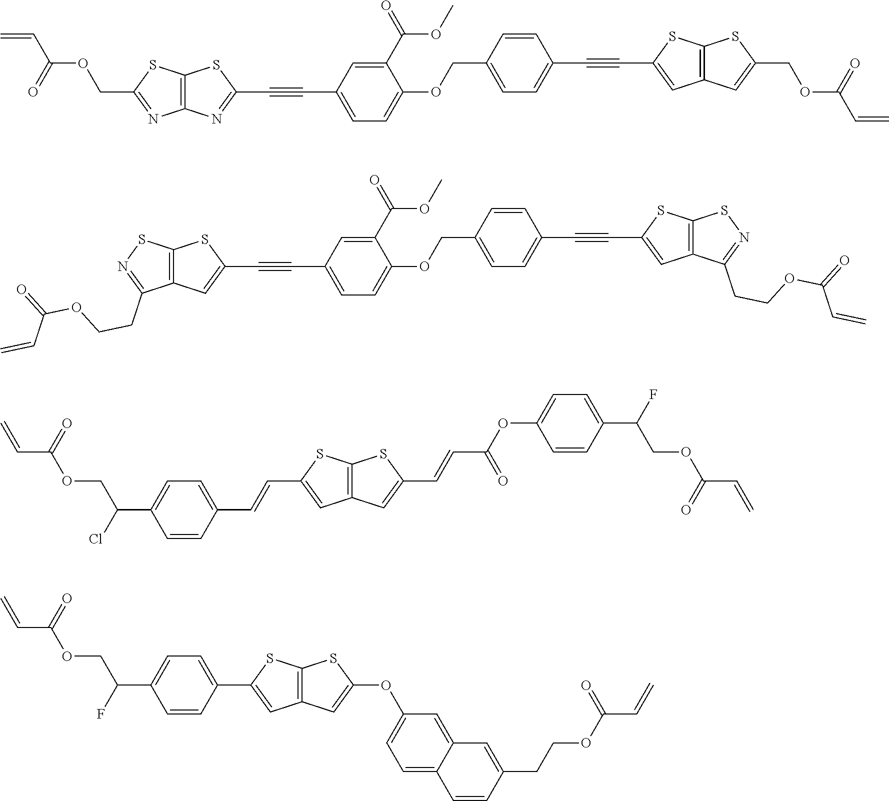

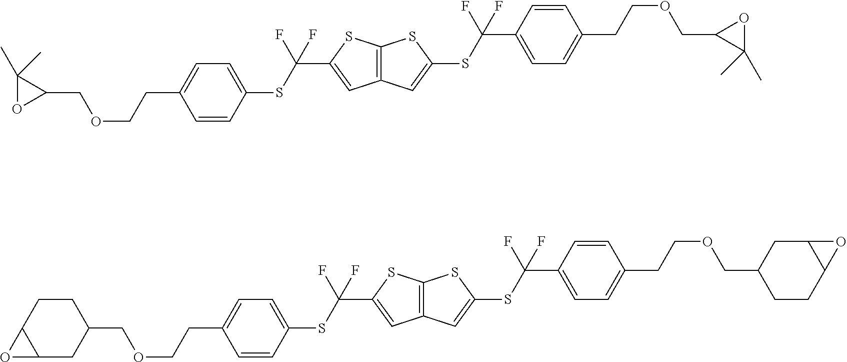

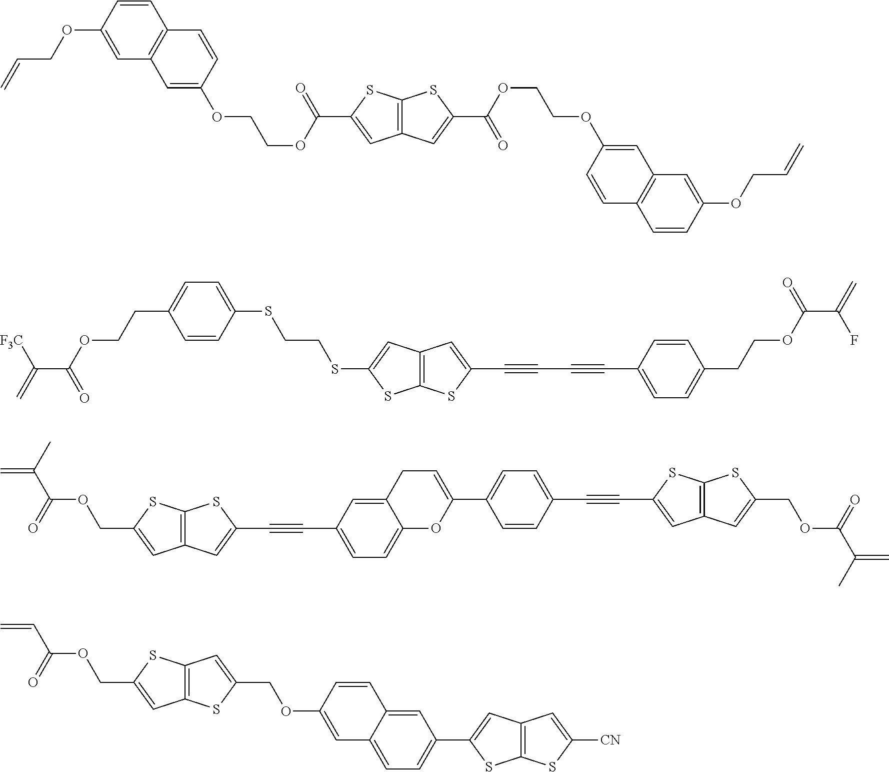

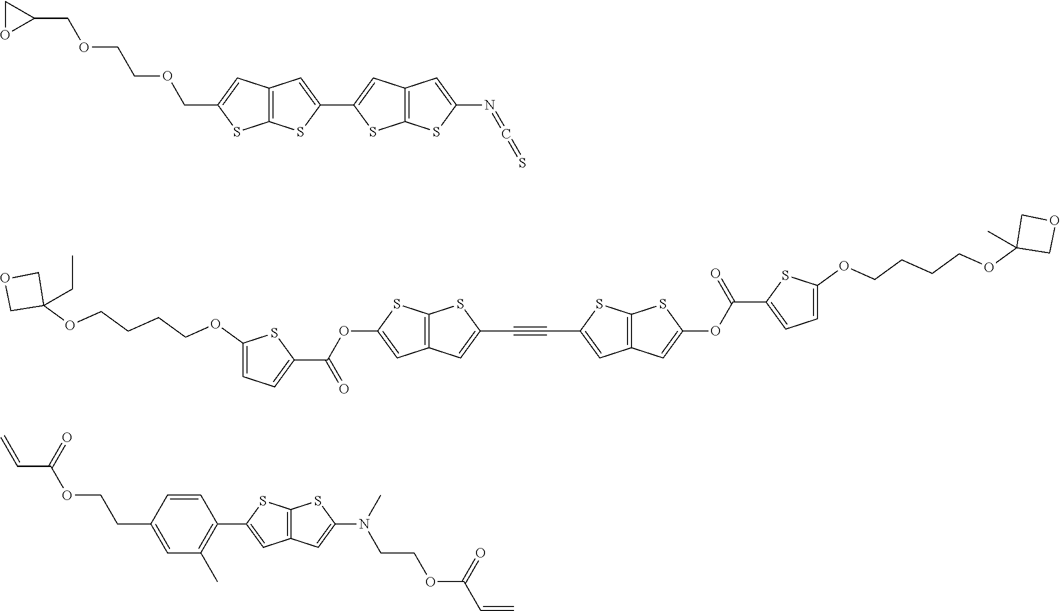 [SMILES: S=C=Nc1cc2cc(-c3cc4cc(COCCOCC5CO5)sc4s3)sc2s1 ; CCC1(OCCCCOc2ccc(C(=O)Oc3cc4cc(C#Cc5cc6cc(OC(=O)c7ccc(OCCCCOC8(C)COC8)s7)sc6s5)sc4s3)s2)COC1 ; C=CC(=O)OCCc1ccc(-c2cc3cc(N(C)CCOC(=O)C=C)sc3s2)c(C)c1]

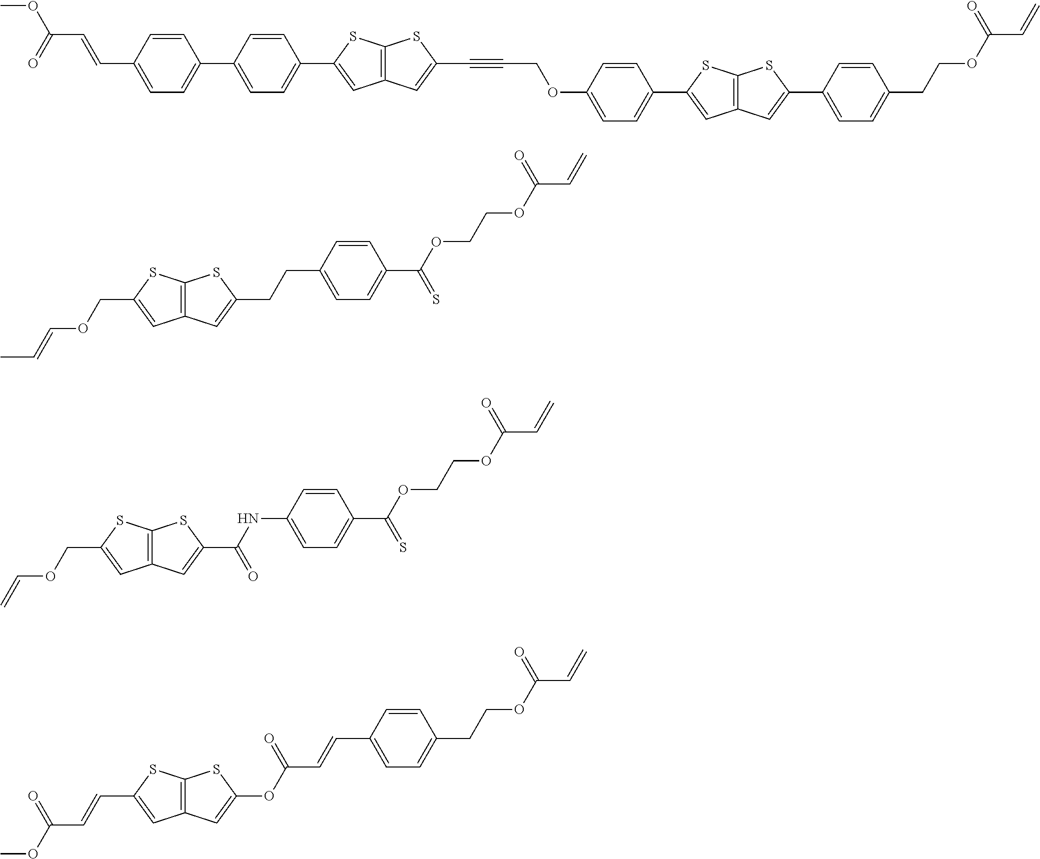

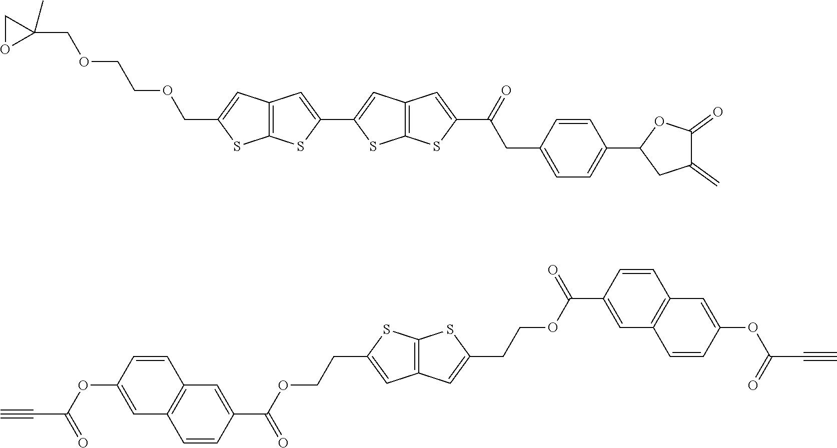

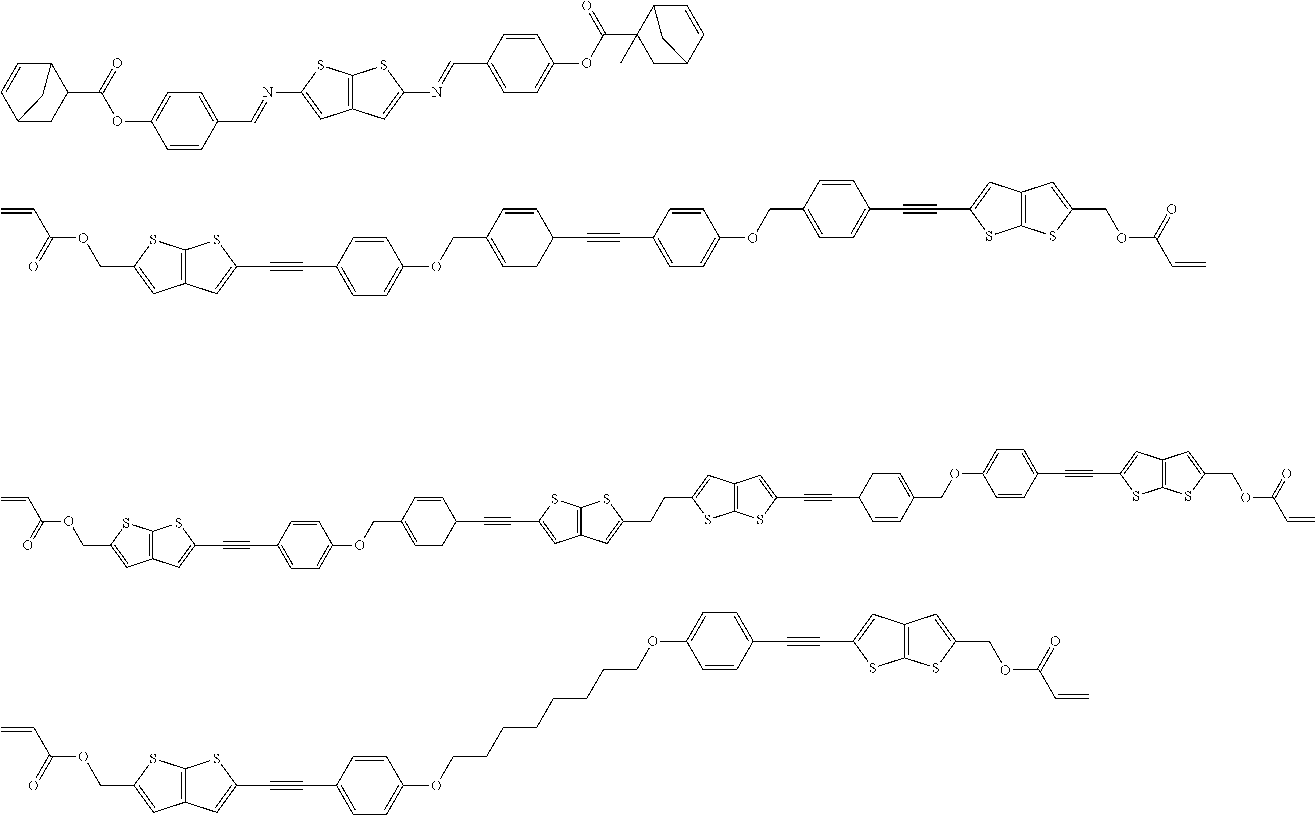

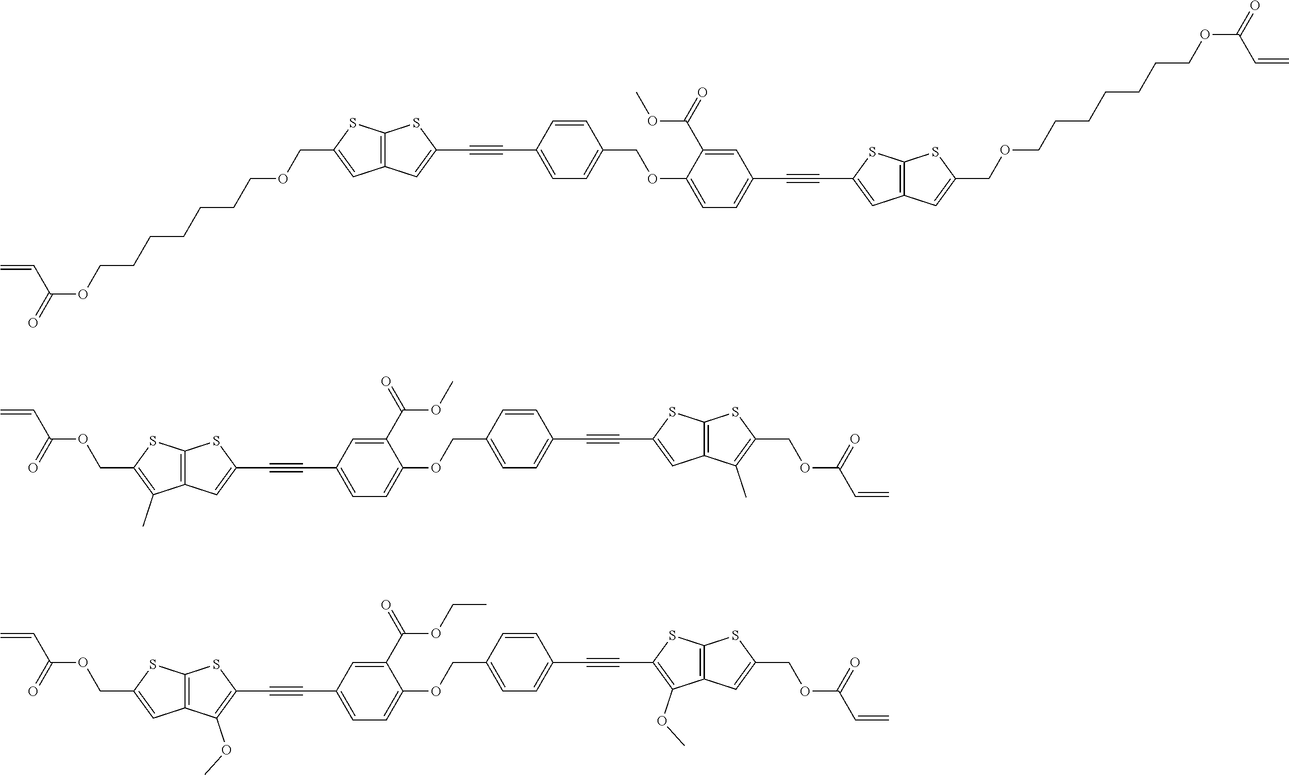

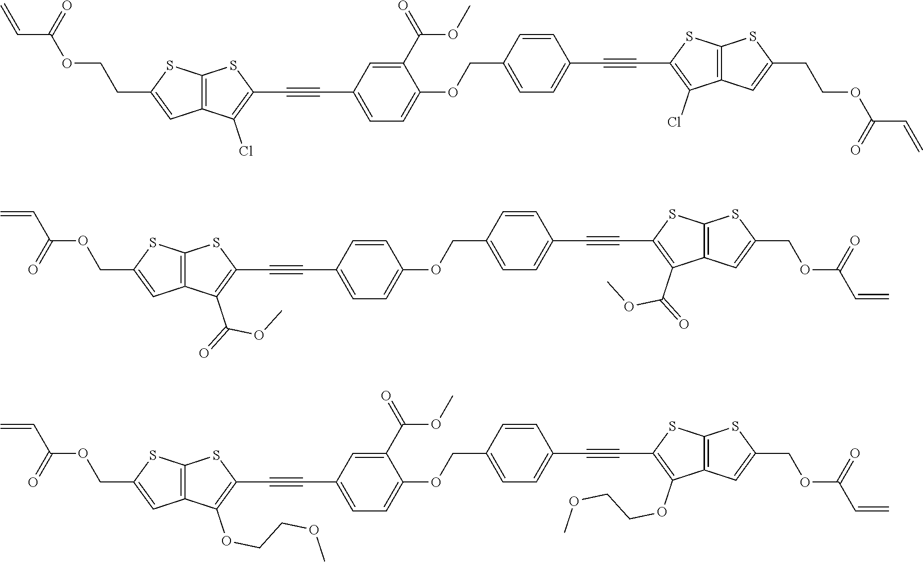

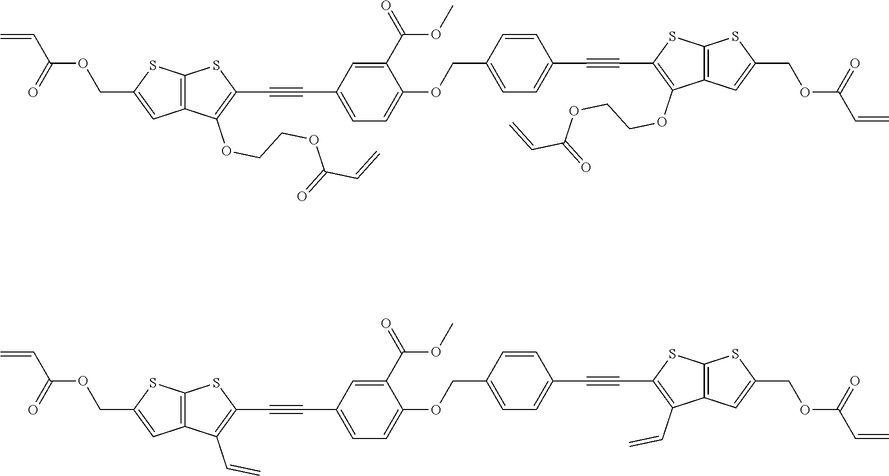

The specific compound may have liquid crystallinity or may not have liquid crystallinity, but preferably has liquid crystallinity.

For the compound exhibiting liquid crystallinity, it is intended that the compound has properties of expressing a mesophase between a crystalline phase (low temperature side) and an isotropic phase (high temperature side) in a case of changing a temperature. As a specific observation method, optical anisotropy and fluidity derived from a liquid crystalline phase can be confirmed by performing an observation using a polarizing microscope while heating the compound or lowering a temperature of the compound with a hot stage system FP90, manufactured by METTLER TOLEDO, or the like.

### [Composition]

### [Specific Compound]

The composition according to the embodiment of the present invention contains a compound represented by Formula (I) (specific compound). The specific compound is as described above.

A content of the specific compound in the composition is not particularly limited, but for example, it is preferably 10% to 100% by mass, more preferably 30% to 99% by mass, and still more preferably 50% to 98% by mass with respect to the total mass of the liquid crystal compound in the composition.

In the composition, the specific compound may be used alone or in combination of two or more types thereof. In a case where two or more types are used, the total content thereof is preferably within the above-described range.

### [Liquid Crystal Compound]

The composition according to the embodiment of the present invention may contain a liquid crystal compound other than the specific compound.

In general, the liquid crystal compound can be classified into a rod-like type and a disk-like type according to the shape thereof. Furthermore, the above-described types of compounds respectively include a low-molecular-weight type compound and a polymer type compound. The term “high-molecular-weight” generally refers to a compound having a degree of polymerization of 100 or more (Polymer Physics-Phase Transition Dynamics, written by Masao Doi, p. 2, published by Iwanami Shoten, 1992).

The liquid crystal compounds are not particularly limited, and any compound may be used. Among these, from the viewpoint that the effect of the present invention is more excellent, a rod-like liquid crystal compound or a disk-like liquid crystal compound (discotic liquid crystal compound) is preferable, and a rod-like liquid crystal compound is more preferable.

In addition, the liquid crystal compound is also preferably a liquid crystal compound having a polymerizable group in the molecule (a polymerizable liquid crystal compound).

Examples of the polymerizable group include an ethylenically unsaturated group and a ring-polymerizable group, and specific examples thereof include a vinyl group, a styryl group, an allyl group, and a substituent selected from the above-described polymerizable group P.

In a case where the liquid crystal compound contains a polymerizable group, the number of polymerizable groups is not particularly limited, but is, for example, one or more, and from the viewpoint of immobilizing the alignment, the liquid crystal compound has more preferably two or more polymerizable groups in one molecule. The upper limit value thereof is, for example, preferably 6 or less and more preferably 3 or less.

The liquid crystal compounds may be used alone or in combination of two or more types thereof.

In a case where two or more liquid crystal compounds are used in combination, any form of a mixture of two or more rod-like liquid crystal compounds, a mixture of two or more disk-like liquid crystal compounds, and a mixture of a rod-like liquid crystal compound and a disk-like liquid crystal compound may be adopted.

In a case where a plurality of types of the liquid crystal compounds are used in combination, it is also preferable that at least one or more of the liquid crystal compounds is a polymerizable liquid crystal compound.

As the liquid crystal compounds, known compounds can be used.

As the rod-like liquid crystal compound, for example, compounds described in [claim 1] of JP1999-513019A (JP-H11-513019A), paragraphs to of JP2005-289980A, WO2019/182129A, and JP2023-003351A, and the like can be suitably used.

In addition, as the disk-like liquid crystal compound, for example, compounds described in paragraphs to of JP2007-108732A and paragraphs to of JP2010-244038A, and the like can be suitably used.

Examples of the rod-like liquid crystal compound include a rod-like nematic liquid crystal compound.

Preferable examples of the rod-like nematic liquid crystal compounds include azomethines, azoxys, cyanobiphenyls, cyanophenyl esters, benzoic acid esters, cyclohexanecarboxylic acid phenyl esters, cyanophenylcyclohexanes, cyano-substituted phenylpyrimidines, alkoxy-substituted phenylpyrimidines, phenyldioxanes, tolans, and alkenylcyclohexylbenzonitriles. As the liquid crystal compound, not only a low-molecular-weight liquid crystal compound but also a high-molecular-weight liquid crystal compound can be used.

It is preferable that the liquid crystal compound has a high refractive index anisotropy Δn, and specifically, the refractive index anisotropy Δn is preferably 0.15 or more, more preferably 0.18 or more, still more preferably 0.22 or more, and particularly preferably 0.25 or more. The upper limit thereof is not particularly limited; however, it is 0.60 or less in many cases.

In addition, by using the specific compound mixed with the liquid crystal compound, the crystallization temperature as a whole can be significantly lowered.

Examples of liquid crystal compound include compounds disclosed in Makromol. Chem., Vol. 190, page 2255 (1989), Advanced Materials, Vol. 5, page 107 (1993), U.S. Pat. Nos. 4,683,327A, 4,983,479A, 5,622,648A, 5,770,107A, WO95/022586A, WO95/024455A, WO97/000600A, WO98/023580A, WO98/052905A, JP1989-272551A (JP-H1-272551A), JP1994-016616A (JP-H6-016616A), JP1995-110469A (JP-H7-110469A), JP1999-080081A (JP-H11-080081A), JP2001-328973A.

In a case where the composition according to the embodiment of the present invention contains a liquid crystal compound, the content of the compound in the composition is not particularly limited, but is preferably 0% to 90% by mass, more preferably 0% to 70% by mass, and still more preferably 0% to 50% by mass with respect to the total mass of the solid content in the composition.

In the composition according to the embodiment of the present invention, the liquid crystal compound may be used alone or in combination of two or more types thereof. In a case where two or more types are used, the total content thereof is preferably within the above-described range.

### [Polymerization Initiator]

The composition preferably contains a polymerization initiator.

The polymerization initiator is preferably a photopolymerization initiator capable of initiating a polymerization reaction by ultraviolet irradiation.

Examples of the photopolymerization initiator include an α-carbonyl compound, acyloin ether, an α-hydrocarbon-substituted aromatic acyloin compound, a polynuclear quinone compound, a phenazine compound, an oxadiazole compound, and a compound having an oxime ester structure.

In a case where the composition according to the embodiment of the present invention includes a polymerization initiator, the content of the polymerization initiator in the composition is not particularly limited, but is preferably 0.1 to 20 mass % and more preferably 1 to 8 mass % with respect to the total mass of the specific compound (in a case where the composition includes liquid crystal compounds, with respect to the total mass of the specific compound and the liquid crystal compounds).

In the composition according to the embodiment of the present invention, one type of polymerization initiator may be used alone, or two or more types thereof may be used. In a case where two or more types are used, the total content thereof is preferably within the above-described range.

The composition according to the embodiment of the present invention may contain a surfactant that contributes to the formation of a stable or rapid liquid crystal phase (for example, a nematic phase, a cholesteric phase).

Examples of the surfactant include a fluorine-containing (meth)acrylate-based polymer, compounds represented by General Formulae (X1) to (X3) disclosed in WO2011/162291A, compounds represented by General Formula (I) disclosed in paragraphs 0082 to 0090 of JP2014-119605A, and compounds disclosed in paragraphs 0020 to 0031 of JP2013-047204A.

Examples of the fluorine-containing (meth)acrylate-based polymer that can be used as a surfactant also include polymers disclosed in paragraphs 0018 to 0043 of JP2007-272185A.

In a case where the composition according to the embodiment of the present invention includes a surfactant, the content of the surfactant is not particularly limited, but is preferably 0.001 to 10 mass % and more preferably 0.05 to 3 mass % with respect to the total mass of the specific compound (in a case where the composition includes liquid crystal compounds, with respect to the total mass of the specific compound and the liquid crystal compounds).

In the composition according to the embodiment of the present invention, one type of surfactant may be used alone, or two or more types thereof may be used. In a case where two or more types are used, the total content thereof is preferably within the above-described range.

### [Chiral Agent]

The composition according to the embodiment of the present invention may contain a chiral agent.

The chiral agent (optically active compound) has a function of causing a helical structure of a cholesteric liquid crystalline phase to be formed. The chiral agent may be selected according to the purpose because a helical twisted direction or a helical pitch of the induced helix varies depending on the compound.

The type of the chiral agent is not particularly limited. The chiral agent may be liquid crystalline or non-liquid crystalline.

The chiral agent generally contains an asymmetric carbon atom, but an axially chiral compound or a planar chiral compound containing no asymmetric carbon atom can also be used as the chiral agent. As the axial asymmetric compound or the planar asymmetric compound, binaphthyl, helicene, paracyclophane, and derivatives thereof are mentioned. The chiral agent may also have a polymerizable group.

In a case where the composition according to the embodiment of the present invention includes a chiral agent, the content of the chiral agent in the composition is not particularly limited, but is preferably 0.1 to 15 mass % and more preferably 1.0 to 10 mass % with respect to the total mass of the specific compound (in a case where the composition includes liquid crystal compounds, with respect to the total mass of the specific compound and the liquid crystal compounds).

In the composition according to the embodiment of the present invention, one type of chiral agent may be used alone, or two or more types thereof may be used.

In a case where two or more types are used, the total content thereof is preferably within the above-described range.

### [Solvent]

The composition according to the embodiment of the present invention may contain a solvent.

The solvent is preferably a solvent capable of dissolving each component formulated in the composition according to the embodiment of the present invention, and examples thereof include chloroform, methyl ethyl ketone, and the like.

In a case where the composition according to the embodiment of the present invention contains the solvent, the content of the solvent in the composition is such an amount that the concentration of solid contents of the composition is preferably 0.5% to 35% by mass and more preferably 1% to 25% by mass.

In the composition according to the embodiment of the present invention, one type of solvent may be used alone, or two or more types thereof may be used. In a case where two or more types are used, the total content thereof is preferably within the above-described range.

### [Other Additives]

The composition according to the embodiment of the present invention may contain other additives in addition to the above-described components.

Examples of the other additives include an antioxidant, an ultraviolet absorber, a sensitizer, a stabilizer, a plasticizer, a chain transfer agent, a polymerization inhibitor, an antifoaming agent, a leveling agent, a thickener, a flame retardant, a dispersant, and a color material such as a dye and a pigment.

### [Refractive Index of Composition]

From the viewpoint that the diffraction efficiency of the obtained film is further increased, the refractive index n of the composition according to the embodiment of the present invention is preferably 1.65 or more, more preferably 1.70 or more, and still more preferably 1.75 or more.

In addition, the refractive index ne in the slow axis direction is preferably 1.80 or more, more preferably 1.90 or more, and still more preferably 2.00 or more. It is noted that the upper limit value thereof is not particularly limited, and is, for example, 3.0 or less.

### [Δn of Composition]

From the viewpoint that the diffraction efficiency of the obtained film is further increased, the refractive index anisotropy Δn of the composition according to the embodiment of the present invention is preferably 0.20 or more, more preferably 0.25 or more, and still more preferably 0.30 or more at a wavelength of 620 nm. It is noted that the upper limit value thereof is not particularly limited, and is, for example, 0.80 or less.

### [Applications of Composition]

The cured product formed of the composition according to the embodiment of the present invention can be used as an optically anisotropic body (optically anisotropic layer).

Hereinafter, the optically anisotropic layer and a manufacturing method thereof will be described.

### <<Example of Embodiment of Optically Anisotropic Layer>>

An example of an embodiment of the optically anisotropic layer consisting of the cured layer of the above-described composition will be described with reference to the drawings.

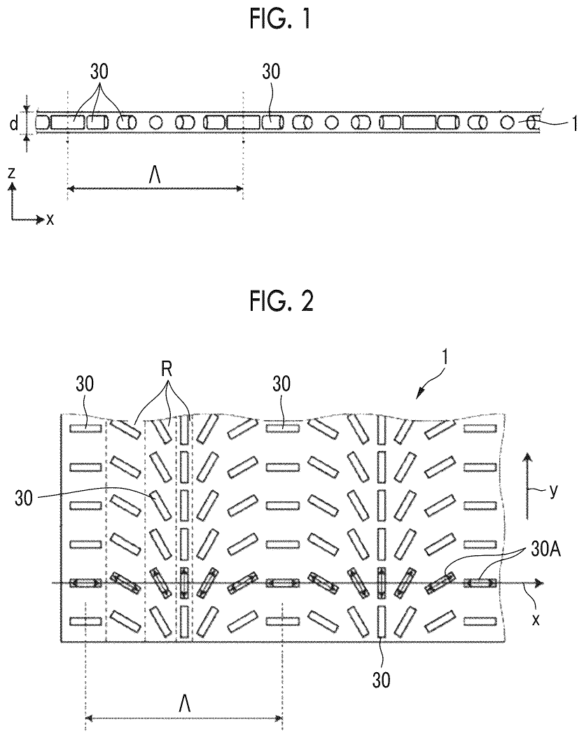

FIGS. 1 and 2 are schematic cross-sectional views of the optically anisotropic layer 1. FIG. 1 is a schematic side view showing the optically anisotropic layer 1, and FIG. 2 is a schematic plan view showing a liquid crystal alignment pattern of the optically anisotropic layer 1 shown in FIG. 1. In the drawings, a sheet surface of the sheet-like optically anisotropic layer 1 is defined as an xy plane, and a thickness direction is defined as a z direction.

As shown in FIG. 1, the optically anisotropic layer 1 has a liquid crystal alignment pattern (length A of one period) in which an orientation of an optical axis derived from the liquid crystal compound 30 is changed while continuously rotating along at least one in-plane direction.

In FIGS. 1 to 4, only the liquid crystal molecules present on one main surface side of the optically anisotropic layer 1 are shown in order to simplify the drawings and clearly show the configuration of the optically anisotropic layer 1. However, the optically anisotropic layer 1 has a structure in which the aligned liquid crystal compounds 30 are laminated same as in an optically-anisotropic layer which is formed using a composition including a normal liquid crystal compound.

Typically, in a case where a value of an in-plane retardation is set as 2/2, the optically anisotropic layer 1 has a function as a general 2/2 plate, that is, a function of imparting a phase difference of a half wavelength, that is, 180° to two linearly polarized light components which are included in light incident into the optically-anisotropic layer and are perpendicular to each other.

As shown in FIG. 2, in a plane of the optically anisotropic layer 1, the optically anisotropic layer 1 has a liquid crystal alignment pattern in which an orientation of an optical axis 30A (hereinafter, may be also referred to as “optical axis 30A”) derived from the liquid crystal compound 30 is changed while continuously rotating in one direction. Here, the one direction in which the optical axis 30A is changed while rotating is set coincidental with a direction of an x-axis in the xy plane. Hereinafter, the one direction in which the optical axis 30A is changed while rotating will be described as an x direction.

The optical axis 30A derived from the liquid crystal compound 30 is an axis having the highest refractive index in the liquid crystal compound 30, that is, a so-called slow axis. As shown in FIG. 1, in a case where the liquid crystal compound 30 is a rod-shaped liquid crystal compound, the optical axis 30A is along a rod-shaped major axis direction.

Specifically, the fact that the direction of the optical axis 30A changes while continuously rotating in the x direction means that angles between the optical axes 30A of the liquid crystal compounds 30 arranged along the x direction and the x direction vary depending on a position in the x direction, and the angles between the optical axes 30A and the x direction gradually change from θ to θ+180° or θ−180° along the x direction. Here, the expression “the angle gradually is changed” means that the angle may be changed at constant angular intervals, or may be changed continuously. A difference between the angles of the optical axes 30A of the liquid crystal compounds 30 adjacent to each other in the x direction is preferably less than or equal to 45°, more preferably less than or equal to 15°, and further preferably a smaller angle.

On the other hand, regarding the liquid crystal compound 30 forming the optically-anisotropic layer 1, the liquid crystal compounds 30 having the same direction of the optical axes 30A are arranged at regular intervals in a y direction perpendicular to the x direction in a plane, that is, a y direction perpendicular to the one direction (x direction) in which the optical axis 30A continuously rotates. In other words, regarding the liquid crystal compound 30 forming the optically anisotropic layer 1, in the liquid crystal compounds 30 arranged in the y direction, angles between the direction of the optical axis 30A and the x direction are the same. In the optically anisotropic layer 1, the length (distance) over which the optical axis 30A of the liquid crystal compound 30 rotates by 180° in the arrow x direction along which the orientation of the optical axis 30A changes while continuously rotating in the liquid crystal alignment pattern of the liquid crystal compound 30 in the plane is defined as a length A of the single period in the liquid crystal alignment pattern. In other words, the length of the single period in the liquid crystal alignment pattern is defined as the distance between θ and θ+180° that is a range of the angle between the optical axis 30A of the liquid crystal compound 30 and the x direction. Specifically, as shown in FIG. 2, a distance of centers in the x direction of two liquid crystal compounds 30 in which the x direction and the direction of the optical axis 30A match each other is defined as the length A of the single period (hereinafter, may be also referred to as “one period A” or “period A”). The liquid crystal alignment pattern of the optically anisotropic layer 1 is a pattern in which the liquid crystal alignment of this single period A is repeated in the x direction.

As described above, in the optically anisotropic layer 1, in the liquid crystal compounds 30 arranged in the y direction, the angles between the optical axes 30A thereof and the x direction in which the orientation of the optical axis of the liquid crystal compound 30 rotates are the same. Regions where the liquid crystal compounds 30 in which the angles between the optical axes 30A and the x direction are the same are arranged in the y direction will be referred to as “regions R”.

In this case, a value of the in-plane retardation (Re) in each region R is a half wavelength of light (hereinafter, referred to as a “target light”) to be diffracted by the optically anisotropic layer, that is, in a case where a wavelength of the target light is 2, the in-plane retardation Re is preferably 2/2. The in-plane retardation is calculated from the product of a refractive index anisotropy Δn of the regions R and the thickness (film thickness) d of the optically anisotropic layer. Here, the difference in refractive index generated by refractive index anisotropy of the region R in the optically anisotropic layer is defined by a difference between a refractive index of a direction of an in-plane slow axis of the region R and a refractive index of a direction orthogonal to the direction of the slow axis. That is, the difference Δn in refractive index due to the refractive index anisotropy of the regions R is the same as a difference between a refractive index of the liquid crystal compound 30 in the direction of the optical axis 30A and a refractive index of the liquid crystal compound 30 in a direction perpendicular to the optical axis 30A in a plane of the region R. That is, the above-described difference Δn in refractive index depends on the liquid crystal compound, and the in-plane retardation of each region R is substantially the same. However, as described above, each region R has a different direction of the optical axis 30A.

In the optically anisotropic layer 1, since the orientation of the optical axis 30A rotates in the plane, it is difficult to measure the in-plane retardation as a whole. However, the in-plane retardation of the optically anisotropic layer 1 can be estimated from the period and the diffraction efficiency.

In a case where circularly polarized light is incident into such an optically anisotropic layer 1, the light is refracted such that the direction of the circularly polarized light is converted.

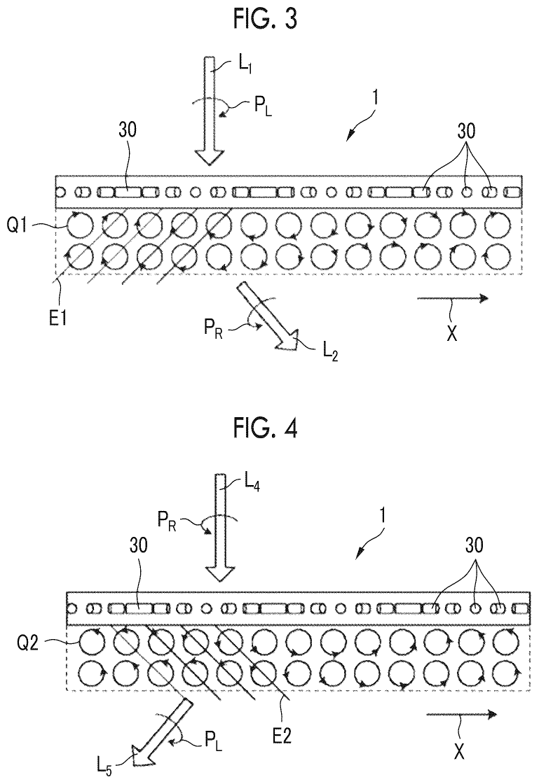

This action is conceptually shown in FIG. 3 using the optically anisotropic layer 1 as an example. It is assumed that the in-plane retardation of the optically anisotropic layer 1 is 22.

In this case, as illustrated in FIG. 3, in a case where an incident ray L₁ that is levorotatory circularly polarized light P_{L} is incident on the optically anisotropic layer 1, the incident ray L₁ passes through the optically anisotropic layer 1, and a phase difference of 180° is imparted to the incident ray L₁. Accordingly, a transmitted ray L² is converted into dextrorotatory circularly polarized light P_{R}.

While the incident ray L₁ is passing through the optically anisotropic layer 1, an absolute phase of the incident ray L₁ changes in accordance with the direction of the optical axis 30A of each liquid crystal compound 30. At this time, since the orientation of the optical axis 30A changes while rotating in the x direction, the amount of change in absolute phase of the incident ray L₁ varies depending on the orientation of the optical axis 30A. Furthermore, the liquid crystal alignment pattern formed in the optically anisotropic layer 1 is a pattern which is periodic in the x direction. Therefore, as shown in FIG. 3, the incident ray L₁ transmitted through the optically anisotropic layer 1 is imparted with an absolute phase Q1 which is periodic in the x direction corresponding to the orientation of each optical axis 30A. As a result, an equiphase surface E1 which is tilted in a direction opposite to the x direction is formed.

Therefore, the transmitted ray L₂ is refracted to be tilted in a direction perpendicular to the equiphase surface E1 and travels in a direction different from a traveling direction of the incident ray L₁. The incident ray L₁ of the levorotatory circularly polarized light P_{L} is converted into the transmitted ray L₂ of the dextrorotatory circularly polarized light P_{R} inclined in the x direction by a predetermined angle with respect to an incidence direction.

Meanwhile, as conceptually illustrated in FIG. 4, in a case where an incident ray L₄ of the dextrorotatory circularly polarized light P_{R} is incident on the optically anisotropic layer 1 having the same in-plane retardation, the incident ray L₄ passes through the optically anisotropic layer 1, and a phase difference of 180° is imparted to the incident ray L₄. Accordingly, the incident ray L₄ is converted into a transmitted ray L₅ of the levorotatory circularly polarized light P_{L}.

While the incident ray L₄ is passing through the optically anisotropic layer 1, an absolute phase of the incident ray L₄ changes in accordance with the direction of the optical axis 30A of each liquid crystal compound 30. At this time, since the orientation of the optical axis 30A changes while rotating in the x direction, the amount of change in absolute phase of the incident ray L₄ varies depending on the orientation of the optical axis 30A. Furthermore, the liquid crystal alignment pattern formed in the optically anisotropic layer 1 is a pattern which is periodic in the x direction. Therefore, as shown in FIG. 4, the incident ray L₄ transmitted through the optically anisotropic layer 1 is imparted with an absolute phase Q2 which is periodic in the x direction corresponding to the orientation of each optical axis 30A.

Here, the incident ray L₄ is right circularly polarized light P_{R}. Therefore, the absolute phase Q2 which is periodic in the x direction corresponding to the direction of the optical axis 30A is opposite to the incident ray L₁ as left circularly polarized light P_{L}. As a result, in the incident ray L₄, an equiphase surface E2 which is tilted in the x direction opposite to that of the incident ray L₁ is formed.

Therefore, the incident ray L₄ is refracted to be tilted in a direction perpendicular to the equiphase surface E2 and travels in a direction different from a traveling direction of the incident ray L₄. In this way, the incident ray L₄ is converted into the transmitted ray L₅ of levorotatory circularly polarized light which is tilted by a predetermined angle in a direction opposite to the x direction with respect to an incidence direction.

As described above, in the optically anisotropic layer 1, the value of the in-plane retardation is preferably half the wavelength of the target light. This is because that, as the value of the in-plane retardation is closer to the half wavelength of the target light, high diffraction efficiency can be obtained in the diffraction of the target light. The in-plane retardation Re (2)=Δn_{λ}×d of the optically anisotropic layer with respect to incident ray having a wavelength in the x direction of λ nm is preferably within a range defined by the following expression and can be appropriately set.

$$0.7 \times ( λ / 2 ) nm \leq Δ {n}_{λ} \times d \leq 1.3 \times ( λ / 2 ) nm$$

Here, by changing the single period A of the liquid crystal alignment pattern formed in the optically anisotropic layer 1, refraction angles of the transmitted rays L₂ and L₅ can be adjusted. Specifically, as the single period A of the liquid crystal alignment pattern decreases, light transmitted through the liquid crystal compounds 30 adjacent to each other more strongly interfere with each other, so that the transmitted rays L₂ and L₅ can be more largely refracted. Furthermore, by reversing a rotation direction of the optical axis 30A of the liquid crystal compound 30 which rotates in the x direction, a refraction direction of the transmitted ray can be reversed. The period Λ is preferably 50 μm or less, more preferably 25 μm or less, and still more preferably 5 μm or less.

It is sufficient that the film thickness d of the optically-anisotropic layer 1 is appropriately set in order to obtain a desired in-plane retardation, but the film thickness d is preferably 1 μm or less, more preferably 0.8 μm or less, and still more preferably 0.5 μm or less. In particular, in a case where the optically anisotropic layer 1 is used as a birefringent mask for forming a photo-alignment pattern, a smaller film thickness d is preferable. As the film thickness d is smaller, a formation accuracy of the photo-alignment pattern can be improved.

The ratio Λ/d of the period Λ to the film thickness d of the optically-anisotropic layer is preferably 1 or more.

The period Λ of the liquid crystal alignment pattern in the optically-anisotropic layer 1 is obtained from a period of light and dark by observing bright and dark period pattern of bright portions and dark portions with a polarizing microscope under a condition of crossed nicols. Twice the period of the observed bright and dark period pattern corresponds to the period Λ of the liquid crystal alignment pattern.

In addition, the film thickness d of the optically-anisotropic layer 1 can be measured by, for example, observing a cross section of the optically-anisotropic layer with a scanning electron microscope.

In the optically anisotropic layer 1, the refractive index anisotropy Δn at a wavelength of 550 nm is preferably 0.21 or more. The upper limit is not particularly limited, and is preferably 0.8 or less.

In addition, it is also preferable that the optically-anisotropic layer can be made to have a substantially wide range for the wavelength of incident ray by imparting a torsion component to the composition or by laminating different phase difference layers. For example, in the optically anisotropic layer, a method of realizing a 2/2 plate having a wide-range pattern by laminating two liquid crystal layers having different twisted directions is disclosed in, for example, JP2014-089476A and can be suitably used in the optically anisotropic layer according to the embodiment of the present invention.

### <Method for Producing Optically Anisotropic Layer 1>

Specific examples of the method for producing the optically anisotropic layer 1 include an aspect in which a step X of bringing a substrate including an alignment film having a predetermined alignment pattern into contact with a composition to form a composition layer on the alignment film on the substrate, and a step Y of subjecting the composition layer to a heat treatment to align the liquid crystal compound, and then subjecting the same to a curing treatment are included.

After the production of the optically anisotropic layer 1, the above-described substrate may be removed from the optically anisotropic layer, or may not be removed. In addition, in the same manner, the above-described alignment film may be removed from the optically anisotropic layer after the production of the optically anisotropic layer 1, or may not be removed.

Hereinafter, specific procedures for the step X and the step Y will be described in detail.

### (Step X)

### Substrate

In the step X, the type of the substrate to be used is not particularly limited, and examples thereof include known substrates (for example, a resin substrate, a glass substrate, a ceramic substrate, a semiconductor substrate, and a metal substrate).

### Alignment Film

An alignment film is disposed on the substrate. In a case where the alignment film is present, the liquid crystal compound 30 is easily aligned in a predetermined liquid crystal alignment pattern in the production of the optically anisotropic layer 1. As described above, the optically anisotropic layer 1 has a liquid crystal alignment pattern in which the orientation of the optical axis 30A (see FIG. 2) derived from the liquid crystal compound 30 is changed while continuously rotating along one in-plane direction (x direction). Therefore, the alignment film is formed such that the optically anisotropic layer can form the liquid crystal alignment pattern.

Various known alignment films can be used. Examples of the alignment film include a rubbed film formed of an organic compound such as a polymer, an obliquely deposited film formed of an inorganic compound, a film having a microgroove, and a film formed by lamination of Langmuir-Blodgett (LB) films formed with a Langmuir-Blodgett's method using an organic compound such as @-tricosanoic acid, dioctadecylmethylammonium chloride, or methyl stearate.

The alignment film by the rubbing treatment can be formed by rubbing the surface of the polymer layer several times in a certain direction with paper or cloth.

As the material used for the alignment film, polyimide, polyvinyl alcohol, a polymer having a polymerizable group described in JP1997-152509A (JP-H9-152509A), and a material used for forming an alignment film described in JP2005-097377A, JP2005-099228A, JP2005-128503A, and the like, can be suitably used.

In addition, as the alignment film, a so-called photo-alignment film obtained by irradiating a photo-alignment material with polarized or non-polarized light to form an alignment film can be suitably used. In a case of being irradiated with polarized light to form the alignment film, the alignment film can be formed by irradiating the photo-alignment material with polarized light from a vertical direction or an oblique direction, and in a case of being irradiated with non-polarized light to obtain the alignment film, the alignment film can be formed by irradiating the photo-alignment material with non-polarized light from an oblique direction.

Examples of the photo-alignment material used in the photo-alignment film include an azo compound described in JP2006-285197A, JP2007-076839A, JP2007-138138A, JP2007-094071A, JP2007-121721A, JP2007-140465A, JP2007-156439A, JP2007-133184A, JP2009-109831A, JP3883848B, and JP4151746B, an aromatic ester compound described in JP2002-229039A, a maleimide- and/or alkenyl-substituted nadiimide compound having a photo-alignable unit described in JP2002-265541A and JP2002-317013A, a photocrosslinking silane derivative described in JP4205195B and JP4205198B, a photocrosslinking polyimide, a photocrosslinking polyamide, and a photocrosslinking ester described in JP2003-520878A, JP2004-529220A, and JP4162850B, and a photodimerizable compound, in particular, a cinnamate compound, a chalcone compound, a coumarin compound, and the like described in JP1997-118717A (JP-H9-118717A), JP1998-506420A (JP-H10-506420A), JP2003-505561A, WO2010/150748A, JP2013-177561A, and JP2014-012823A. Among these, an azo compound, a photocrosslinking polyimide, a photocrosslinking polyamide, a photocrosslinking ester, a cinnamate compound, a chalcone compound, or the like can be suitably used.

The thickness of the alignment film is not particularly limited and may be appropriately set according to the material for forming the alignment film such that a required alignment function can be obtained.

The thickness of the alignment film is preferably 0.01 to 5 μm and more preferably 0.05 to 2 μm.

The method of forming the alignment film is not particularly limited, and various known methods can be used according to the material for forming the alignment film.

From the viewpoint that the alignment pattern of the optically anisotropic layer 1 is more easily formed, a photo-alignment film is preferable, which is obtained by irradiating the photo-alignment material with polarized or non-polarized light to form an alignment film. A method described in [0078] to [0080] of WO2020/022496A can be suitably applied.

### Procedure of Step X

A method of bringing a substrate including an alignment film having a predetermined alignment pattern (hereinafter, also referred to as “substrate with an alignment film”) into contact with the composition is not particularly limited, and examples thereof include a method of applying the composition onto the alignment film on the substrate and a method of immersing the above-described substrate with the alignment film in the composition.

After the substrate with an alignment film is brought into contact with the composition, a drying treatment may be performed as necessary to remove a solvent from the composition layer disposed on the alignment film on the substrate.

### (Step Y)

The step Y is a step of subjecting the composition layer to a heat treatment to align the liquid crystal compound, and then subjecting the same to a curing treatment. By subjecting the composition layer to a heat treatment, the liquid crystal compound is aligned to form a liquid crystal phase. For example, in a case where the composition layer contains a chiral agent, a cholesteric liquid crystalline phase is formed.

Conditions of the heat treatment are not particularly limited, and optimum conditions are selected depending on the type of the liquid crystal compound.

The method of the curing treatment is not particularly limited, and examples thereof include photo-curing treatment and thermosetting treatment. Above all, a light irradiation treatment is preferable, and an ultraviolet irradiation treatment is more preferable.

A light source such as an ultraviolet lamp is used for ultraviolet irradiation.

The cured product that is obtained by the above treatment corresponds to a layer that is obtained by immobilizing a liquid crystal phase. In particular, in a case where the composition contains a chiral agent, a layer is formed in which a cholesteric liquid crystalline phase is immobilized.

These layers do not need to exhibit liquid crystallinity anymore. More specifically, for example, as a state in which the cholesteric liquid crystalline phase is “immobilized,” the most typical and preferred aspect is a state in which the alignment of the liquid crystal compound, which is the cholesteric liquid crystalline phase, is retained. More specifically, it is preferably a state in which within a temperature range of usually 0° C. to 50° C., or −30° C. to 70° C. under the more severe conditions, no fluidity is exhibited in the layer, no changes in alignment form occur due to an external field or an external force, and a fixed alignment form can be kept stably and continuously.

### <<Modification Example of Optically Anisotropic Layer>>

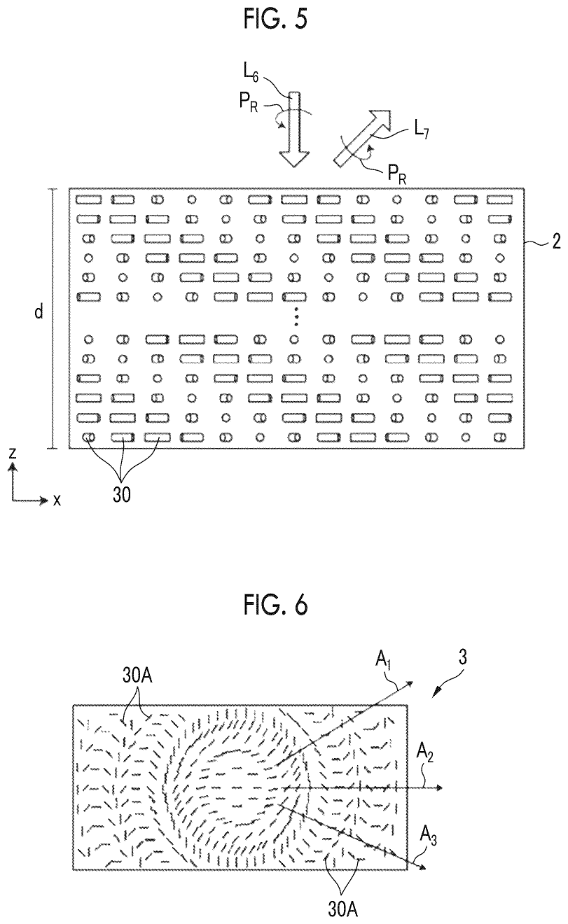

The optically anisotropic layer 2 shown in FIG. 5 is an optically anisotropic layer in which the liquid crystal compound 30 is cholesterically aligned in a thickness direction.

It is known that a cholesteric liquid crystalline phase exhibits selective reflectivity at a specific wavelength.

The center wavelength λ of selective reflection (selective reflection center wavelength λ) depends on a pitch P (=helical period) of a helical structure in the cholesteric liquid crystalline phase and satisfies a relationship of λ=n×P with an average refractive index n of the cholesteric liquid crystalline phase. Therefore, the selective reflection center wavelength can be adjusted by adjusting the pitch of the helical structure.

The cholesteric liquid crystalline phase exhibits selective reflectivity with respect to either levorotatory or dextrorotatory circularly polarized light at a specific wavelength. Whether the reflected light is dextrorotatory circularly polarized light or levorotatory circularly polarized light depends on the twisted direction (sense) of the helix of the cholesteric liquid crystalline phase. Regarding the selective reflection of circularly polarized light by the cholesteric liquid crystalline phase, dextrorotatory circularly polarized light is reflected in a case where the helical twisted direction of the cholesteric liquid crystalline phase is right, and levorotatory circularly polarized light is reflected in a case where the helical twisted direction is left.

In addition, a half-width Δλ (nm) of a selective reflection range (circularly polarized light reflection range) where selective reflection is exhibited depends on Δn of the cholesteric liquid crystalline phase and the helical pitch P and complies with a relationship of Δλ=Δn×P. Therefore, the width of the selective reflection range can be controlled by adjusting Δn.

That is, the optically anisotropic layer 2 exhibits a function of selectively reflecting light in a predetermined wavelength range in specific circularly polarized light (dextrorotatory circularly polarized light or levorotatory circularly polarized light).

On the other hand, since the alignment pattern of the optical axis 30A in the in-plane direction of the optically anisotropic layer 2 is the same as the alignment pattern in the optically anisotropic layer 1 shown in FIG. 1, the same effect as the optically anisotropic layer 1 is exhibited. That is, the optically anisotropic layer 2 has the same action as the optically anisotropic layer 1 described above, of changing the absolute phase of the incident light and bending the light in a predetermined direction. Therefore, the optically anisotropic layer 2 has both the action of bending the incident ray to a direction different from the incidence direction and the action of the above-described cholesteric alignment, and reflects light at an angle in a predetermined direction with respect to the reflection direction of specular reflection.

For example, the optically anisotropic layer 2 is designed such that the cholesteric liquid crystalline phase of the optically anisotropic layer 2 reflects dextrorotatory circularly polarized light. In this case, as shown in FIG. 5, in a case where light L₆ which is dextrorotatory circularly polarized light P_{R} is vertically incident into the main surface of the optically anisotropic layer 2, that is, along the normal line, reflected light L₇ which travels in a direction having a tilt with respect to the normal direction is generated. That is, the optically anisotropic layer 2 functions as a reflective type diffraction grating.

In the liquid crystal alignment pattern of the optically anisotropic layer shown in FIGS. 1 to 5, the optical axes 30A of the liquid crystal compound 30 continuously rotate only along the x direction in the plane.

However, in the optically anisotropic layer according to the embodiment of the present invention, various configurations can be used as long as the optical axis 30A of the liquid crystal compound 30 continuously rotates along one direction.

FIG. 6 is a schematic plan view of the optically anisotropic layer 3 of the design change example. In FIG. 6, the liquid crystal alignment pattern is shown by the optical axis 30A of the liquid crystal compound. The optically anisotropic layer 3 has a liquid crystal alignment pattern that regions in which the optical axes 30A have the same orientation are provided concentrically, and that the one direction in which the orientation of optical axis 30A is changed while continuously rotating is provided radially from the center of the optically anisotropic layer 3.

In the optically anisotropic layer 3, the orientations of the optical axes 30A are changed while continuously rotating along a large number of directions from the center of the optically anisotropic layer 3 toward the outside, for example, a direction indicated by an arrow A₁, a direction indicated by an arrow A₂, a direction indicated by an arrow A₃, and the like.

In circularly polarized light incident into the optically anisotropic layer 3 having the above-described liquid crystal alignment pattern, an absolute phase changes depending on individual local regions having different orientations of optical axes of the liquid crystal compound 30. In this case, the amount of change in absolute phase in each of the local regions varies depending on the orientations of the optical axes of the liquid crystal compound 30 into which circularly polarized light is incident.

This way, in the optically anisotropic layer 3 having the concentric circular liquid crystal alignment pattern, that is, the liquid crystal alignment pattern in which the optical axis changes while continuously rotating in a radial shape, transmission of incident ray can be allowed as diverging light or converging light depending on the rotation direction of the optical axis of the liquid crystal compound 30 and the direction of circularly polarized light to be incident.

That is, by setting the liquid crystal alignment pattern of the optically anisotropic layer in a concentric circular shape, the optically anisotropic layer exhibits, for example, a function as a convex lens or a concave lens.

Here, in a case where the liquid crystal alignment pattern of the optically anisotropic layer is concentric circular such that the optically anisotropic layer functions as a convex lens, it is preferable that the length of the single period Λ over which the optical axis rotates 180° in the liquid crystal alignment pattern gradually decreases from the center of the optically anisotropic layer 3 toward the outer direction of the one direction in which the optical axis continuously rotates. The refraction angle of light with respect to an incidence direction increases as the length of the single period Λ in the liquid crystal alignment pattern decreases. Therefore, the light focusing power of the optically anisotropic layer 3 can be further improved and the performance as a convex lens can be improved by gradually shortening the single period A in the liquid crystal alignment pattern from the center of the optically anisotropic layer 3 toward the outer direction of the one direction in which the optical axis continuously rotates.

In addition, depending on the uses of the laminate such as a concave lens, it is preferable that the length of the single period Λ over which the optical axis rotates 180° in the liquid crystal alignment pattern gradually decreases from the center of the optically anisotropic layer 3 toward the outer direction of the one direction by reversing the direction in which the optical axis continuously rotates. The refraction angle of light with respect to an incidence direction increases as the length of the single period Λ in the liquid crystal alignment pattern decreases. Accordingly, the length of the single period Λ in the liquid crystal alignment pattern gradually decreases from the center of the optically anisotropic layer 3 toward the outer direction in the in-plane direction in which the optical axis continuously rotates. As a result, the light diverging power of the optically anisotropic layer 3 can be improved, and the performance as a concave lens can be improved.

For example, in a case where the optically anisotropic layer is used as a concave lens, it is also preferable that the turning direction of incident circularly polarized light is reversed.

Conversely, the length of the single period Λ in the concentric circular liquid crystal alignment pattern may gradually increase from the center of the optically anisotropic layer 3 toward the outer direction of the one direction in which the optical axis continuously rotates.

Furthermore, depending on the uses of the optically anisotropic layer such as a case where it is desired to provide a light amount distribution in the transmitted light, a configuration in which regions having partially different lengths of the single periods A in the one direction in which the optical axis continuously rotates are provided can also be used instead of the configuration in which the length of the single period Λ gradually changes in the one direction in which the optical axis continuously rotates.

In addition, the light emitting element may include: an optically-anisotropic layer in which the single period Λ is homogeneous over the entire surface; and an optically-anisotropic layer in which regions having different lengths of the single periods A are provided.

In this way, the configuration of changing the length of the single period Λ over which the optical axis rotates 180° in the one direction in which the optical axis continuously rotates can also be used in the configuration shown in FIGS. 1 to 4 in which the optical axis 30A of the liquid crystal compound 30 changes while continuously rotating only in the one direction of the x direction.

For example, by gradually decreasing the single period Λ of the liquid crystal alignment pattern in the x direction, an optically anisotropic layer which transmits light so as to be gathered can be obtained. In addition, by reversing the direction over which the optical axis in the liquid crystal alignment pattern rotates 180°, an optically anisotropic layer which transmits light so as to be diffused only in the x direction can be obtained. By reversing the turning direction of incident circularly polarized light, an optically anisotropic layer that allows transmission of light to be diffused only in the arrow X direction can be obtained.

Furthermore, depending on the application of the optically anisotropic layer such as a case where it is desired to provide a light amount distribution in the transmitted ray, a configuration in which regions having partially different lengths of the single periods A in the x direction are provided can also be used instead of the configuration in which the length of the single period Λ gradually changes in the x direction.

### [Optical Element]

An optical element according to an embodiment of the present invention includes the above-described optically anisotropic layer (optically anisotropic body).

The application of the optical element is not particularly limited, but the optical element can be used for various uses where transmission of light in a direction different from an incidence direction is allowed, for example, an optical path changing member, a light collecting element, a light diffusing element to a predetermined direction, a diffraction element, or the like in an optical apparatus.

Among these, preferred examples of the application include a light guide element. The light guide element typically includes a light guide plate and a diffraction element that is disposed on the light guide plate (preferably, is disposed to be spaced from the light guide plate). The optical element according to the embodiment of the present invention is suitably used as a diffraction element.

### EXAMPLES

Hereinafter, the present invention will be described in more detail based on Examples. The materials, the amounts of materials used, the proportions, the treatment details, the treatment procedure, and the like shown in Examples below may be appropriately modified as long as the modifications do not depart from the spirit of the present invention. Accordingly, the scope of the present invention should not be construed as being limited to Examples shown below.

The abbreviations used in the following description are as follows.
DMAc: dimethylacetamide
THF: tetrahydrofuran
MeOH: methanol
DMF: N,N-dimethylformamide

### Synthesis of Specific Compound

### Synthesis Example 1: Synthesis of Compound A-1

The compound A-1 was synthesized according to the following scheme. It is noted that a compound 7 was synthesized according to WO2019/182129A.

TMS indicates a trimethylsilyl group (—Si(CH₃)₃).

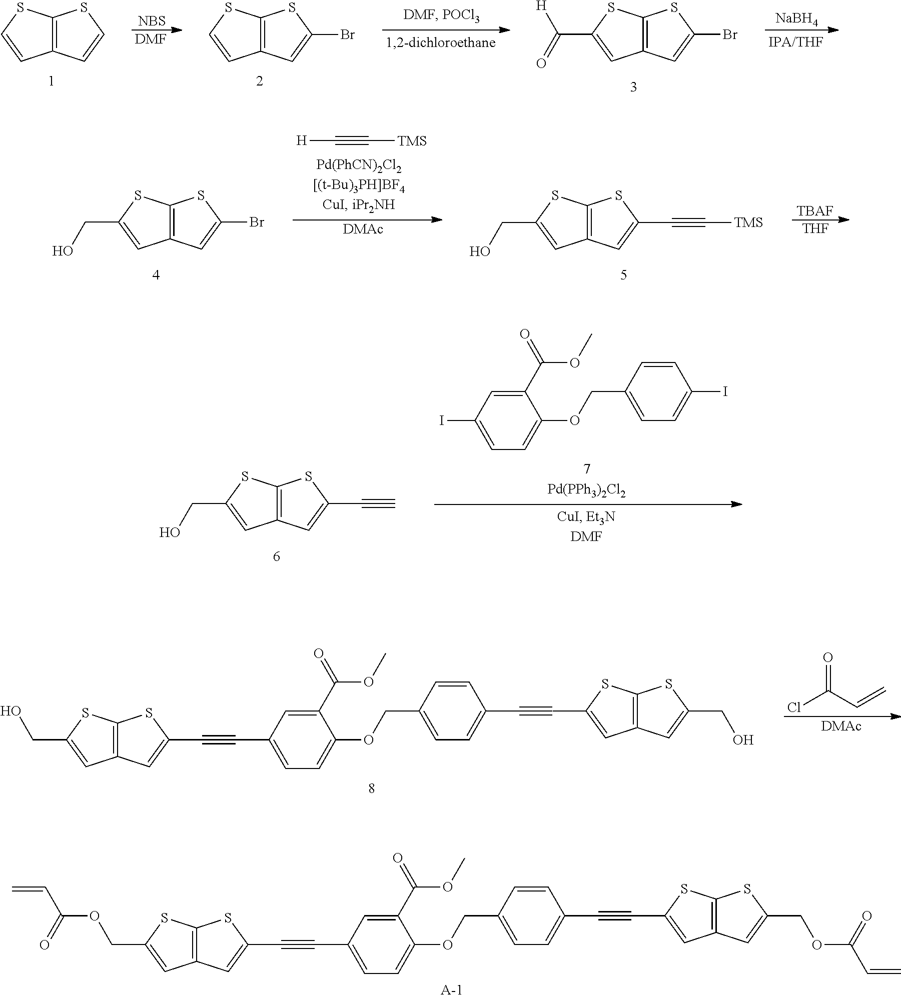

### (1) Synthesis of Compound 4

The thieno[2,3-b]thiophene (compound 1: 6.0 g, 42.8 mmol) was dissolved in DMF (60.0 mL) and cooled to 0° C. Subsequently, N-bromosuccinimide (7.62 g, 42.8 mmol) was added thereto, the temperature was raised to room temperature, and the mixture was stirred for 1 hour. Dichloromethane (120 mL) and water (60 mL) were added thereto, and the mixture was stirred, and then the aqueous phase was removed. The obtained organic layer was sequentially washed with a 1 M lithium chloride aqueous solution, water, and a saline solution, and then dried over magnesium sulfate. After filtering the organic layer, the solvent was distilled off under reduced pressure. The resulting residue was purified by flash column chromatography to obtain a compound 2.

Under a nitrogen atmosphere, DMF (40.3 mL) was cooled to 0° C. Subsequently, phosphorus oxychloride (36.1 mL) was added dropwise thereto, and the mixture was stirred for 1 hour. After dropwise addition of a 1,2-dichloroethane (57.2 mL) solution of the compound 2 thereto, the temperature was raised to 90° C. After stirring for 5 hours, the reaction solution was added to water (400 mL) at 0° C. A 50% sodium hydroxide aqueous solution was added thereto until the pH reached 7.0, and then extraction with dichloromethane was performed. The obtained organic layer was dried over magnesium sulfate, and filtered, and then the solvent was distilled off under reduced pressure. The resulting residue was purified by flash column chromatography to obtain a compound 3.

Under a nitrogen atmosphere, sodium borohydride (1.34 g, 35.5 mmol) was added to isopropanol (101 mL). Subsequently, a THF (53.8 mL) solution of the compound 3 was added dropwise thereto, and the mixture was stirred for 1 hour. After cooling to 0° C., 2N hydrochloric acid and dichloromethane were added thereto, and the mixture was stirred, and then the aqueous phase was removed. The obtained organic layer was sequentially washed with a sodium bicarbonate solution and then a saline solution, followed by being dried over magnesium sulfate. The organic layer was filtered, and the solvent was distilled off under reduced pressure to obtain a compound 4 (8.0 g, 3-step yield: 75.0%).

### (2) Synthesis of Compound 5

Under a nitrogen atmosphere, the compound 4 (8.00 g, 20.4 mmol) was dissolved in DMAc (80 mL), and N,N-diisopropylethylamine (11.4 mL) and trimethylsilyl acetylene (4.04 mL, 28.6 mmol) were added thereto. Thereafter, bis(benzonitrile) palladium (II) dichloride (297 mg), tri-tert-butylphosphonium tetrafluoroborate (432 mg), and copper iodide (190 mg) were added thereto, and the mixture was stirred at 60° C. for 3 hours. The obtained solution was cooled to room temperature, and ethyl acetate (60 mL) was added thereto. Subsequently, the mixture was sequentially washed with a hydrochloric acid solution, water, and a saline solution. The obtained organic layer was dried over magnesium sulfate, and the organic layer was filtered. The solvent was distilled off under reduced pressure, and the resulting residue was purified by flash column chromatography, thereby obtaining a compound 5 (6.48 g, yield: 74.1%).

### (3) Synthesis of Compound 6

The compound 5 (6.48 g, 24.3 mmol) and acetic acid (1.53 mL, 26.8 mmol) were dissolved in THF (32.4 mL), and a 1 mol/L THF solution of tetra-n-butylammonium fluoride (TBAF) (26.8 mL, 26.8 mmol) was added dropwise thereto under ice cooling. After stirring at room temperature for 1 hour, ethyl acetate (48.6 mL) was added thereto, and the mixture was sequentially washed with hydrochloric acid, a sodium bicarbonate solution, and then a saline solution. The obtained organic layer was dried over magnesium sulfate, and the organic layer was filtered. The solvent was distilled off under reduced pressure, and the resulting residue was purified by flash column chromatography, thereby obtaining a compound 6 (3.12 g, yield: 66.0%).

### (4) Synthesis of Compound 8

Under a nitrogen atmosphere, the compound 7 (1.5 g, 3.04 mmol) was dissolved in DMF (15.0 mL), and triethylamine (4.23 mL), triphenylphosphine (60 mg), and tetrabutylammonium bromide (98 mg) were added thereto. Subsequently, bis(triphenylphosphine) palladium (II) dichloride (43 mg) and copper iodide (43 mg) were added thereto, the compound 6 (1.77 g, 9.11 mmol) was added thereto, and the mixture was stirred at 60° C. for 2 hours. The obtained solution was cooled to room temperature, ethyl acetate and THF were added thereto, and the mixture was stirred. The obtained organic layer was sequentially washed with a hydrochloric acid solution, a sodium bicarbonate solution, and then a saline solution. The obtained organic layer was dried over magnesium sulfate, and the organic layer was filtered. The solvent was distilled off under reduced pressure, and the resulting residue was purified by flash column chromatography, and then crystallized with THE and MeOH to obtain a compound 8 (1.80 g, yield: 94.6%).

### (5) Synthesis of Compound A-1

The compound 8 (1.80 g, 2.87 mmol) was dissolved in DMAc (30.6 mL), acryloyl chloride (1.39 mL, 17.2 mmol) was added dropwise thereto, and the mixture was stirred at room temperature for 2 hours.

Chloroform was added thereto, and the mixture was stirred, and then the aqueous phase was removed. The obtained organic layer was sequentially washed with water, a sodium bicarbonate solution, and then a saline solution. The obtained organic layer was dried over sodium sulfate decahydrate, and filtered. The solvent was distilled off under reduced pressure, and the resulting residue was purified by flash column chromatography. The obtained compound was crystallized with THF and MeOH to obtain a compound A-1 (2.01 g, yield: 95.2%).

¹H-NMR of compound A-1 (CDCl₃): δ=3.93 (s, 3H), 5.23 (s, 2H), 5.38 (s, 4H), 5.88 (dd, 2H), 6.15 (dd, 2H), 6.47 (d, 2H), 6.99 (d, 1H), 7.24 (d, 2H), 7.35 (d, 2H), 7.49 (d, 2H), 7.56 (d, 2H), 7.59 (dd, 1H), 8.03 (d, 1H)

### Synthesis Example 2: Synthesis of Compound A-2

The compound A-2 was synthesized according to the following scheme. It is noted that a compound 10 was synthesized according to WO2019/182129A.

TMS indicates a trimethylsilyl group (—Si(CH₃)₃).

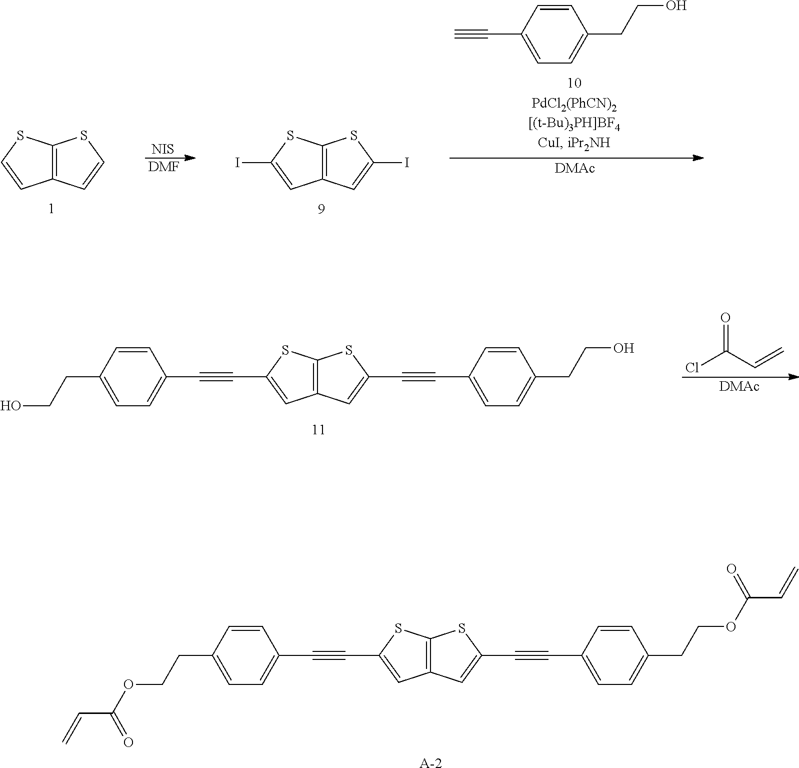

### (1) Synthesis of Compound 9

The compound 1 (1.00 g, 7.13 mmol) was dissolved in DMF (10.0 mL) and cooled to 0° C. N-iodosuccinimide (NIS, 2.6 g, 14.6 mmol) was added thereto, the temperature was raised to 80° C., and the mixture was stirred for 5 hours. Water (10 mL) and dichloromethane (10 mL) were added thereto, and the mixture was stirred, and then the aqueous phase was removed. The obtained organic layer was sequentially washed with a 1 M lithium chloride aqueous solution, water, and then a saline solution. The obtained organic layer was dried over sodium sulfate decahydrate, and the organic layer was filtered. The solvent was distilled off under reduced pressure, and the resulting residue was purified by flash column chromatography. A compound 9 (2.18 g, yield: 78.0%) was obtained.

### (2) Synthesis of Compound 11

Under a nitrogen atmosphere, the compound 9 (1.00 g, 2.55 mmol) and the compound 10 (0.82 g, 5.61 mmol) were dissolved in DMAc (10.0 mL), and N,N-diisopropylethylamine (1.43 mL) was added thereto. Thereafter, bis(benzonitrile) palladium (II) dichloride (37 mg), tri-tert-butylphosphonium tetrafluoroborate (54 mg), and copper iodide (24 mg) were added thereto, and the mixture was stirred at 60° C. for 4 hours. The obtained solution was cooled to room temperature, and ethyl acetate (10 mL) and THF (10 mL) were added thereto. Subsequently, the mixture was sequentially washed with a hydrochloric acid solution, water, and a saline solution. The obtained organic layer was dried over magnesium sulfate, and the organic layer was filtered. The solvent was distilled off under reduced pressure, and the resulting residue was purified by flash column chromatography, thereby obtaining a compound 11 (0.59 g, yield: 54.0%).

### (3) Synthesis of Compound A-2

The compound 11 (0.10 g, 0.23 mmol) was dissolved in DMAc (1.7 mL), acryloyl chloride (0.11 mL, 1.40 mmol) was added dropwise thereto, and the mixture was stirred at room temperature for 2 hours.

Chloroform (6.5 mL) was added thereto, and the mixture was sequentially washed with water, a sodium bicarbonate solution, and then a saline solution. The obtained organic layer was dried over sodium sulfate decahydrate, and the organic layer was filtered. The solvent was distilled off under reduced pressure, and the resulting residue was purified by flash column chromatography. The obtained compound was crystallized with THF and MeOH to obtain a compound A-2 (0.11 g, yield: 87.8%).

¹H-NMR of compound A-2 (CDCl₃): δ=3.01 (dd, 4H), 4.39 (dd, 4H), 5.84 (dd, 2H), 6.11 (dd, 2H), 6.39 (dd, 2H), 7.24 (d, 4H), 7.35 (s, 2H), 7.48 (d, 4H)

### Synthesis Example 3: Synthesis of Compound A-3

The compound A-3 was synthesized according to the following scheme. The compound A-3 was synthesized by the same procedure as in (1) to (5) of <Synthesis Example 1: synthesis of compound A-1>, except that the compound 14 was used.

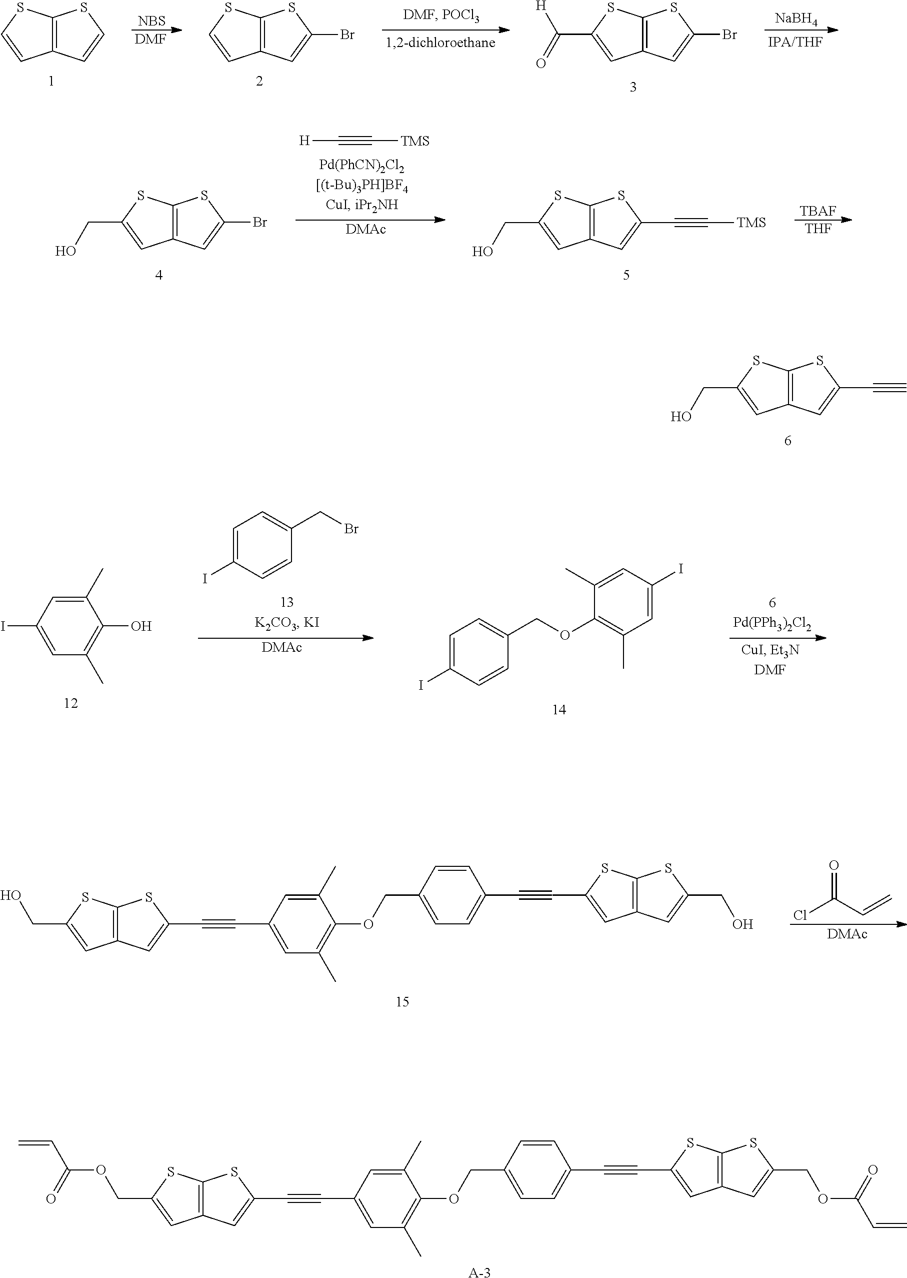

### (1) Synthesis of Compound 14

4-iodo-2,6-dimethylphenol (compound 12: 3.00 g, 12.1 mmol), 4-iodobenzyl bromide (compound 13: 3.68 g, 12.1 mmol), potassium carbonate (1.92 g, 13.9 mmol), and potassium iodide (0.20 g, 1.21 mmol) were dissolved in DMAc (15.0 mL), and the mixture was stirred at 45° C. for 30 minutes and then stirred at 55° C. for 3 hours. After temperature was decreased to room temperature, MeOH (5 mL) was added thereto, and the mixture was stirred for 10 minutes. Subsequently, a mixed solvent of water and MeOH (60 mL) was added dropwise thereto, and water (60 mL) was added dropwise thereto, and the mixture was stirred for 1 hour. The reaction solution was filtered to obtain a compound 14 (5.37 g, yield: 95.7%).

### (2) Compound A-3

The ¹H-NMR of the obtained compound A-3 (CDCl₃) is shown below.

¹H-NMR of compound A-3 (CDCl₃): δ=6.05 (s, 6H), 4.85 (s, 2H), 5.38 (t, 4H), 5.90 (dd, 2H), 6.17 (dd, 2H), 6.49 (dd, 2H), 7.23 (d, 2H), 7.24 (d, 2H), 7.35 (d, 2H), 7.45 (d, 2H), 7.57 (d, 2H)

### Synthesis Example 4: Synthesis of Compound A-4

The compound A-4 was synthesized according to the following scheme. The compound A-4 was synthesized by the same procedure as in (1) to (5) of <Synthesis Example 1: synthesis of compound A-1>, except that the compound 17 was used.

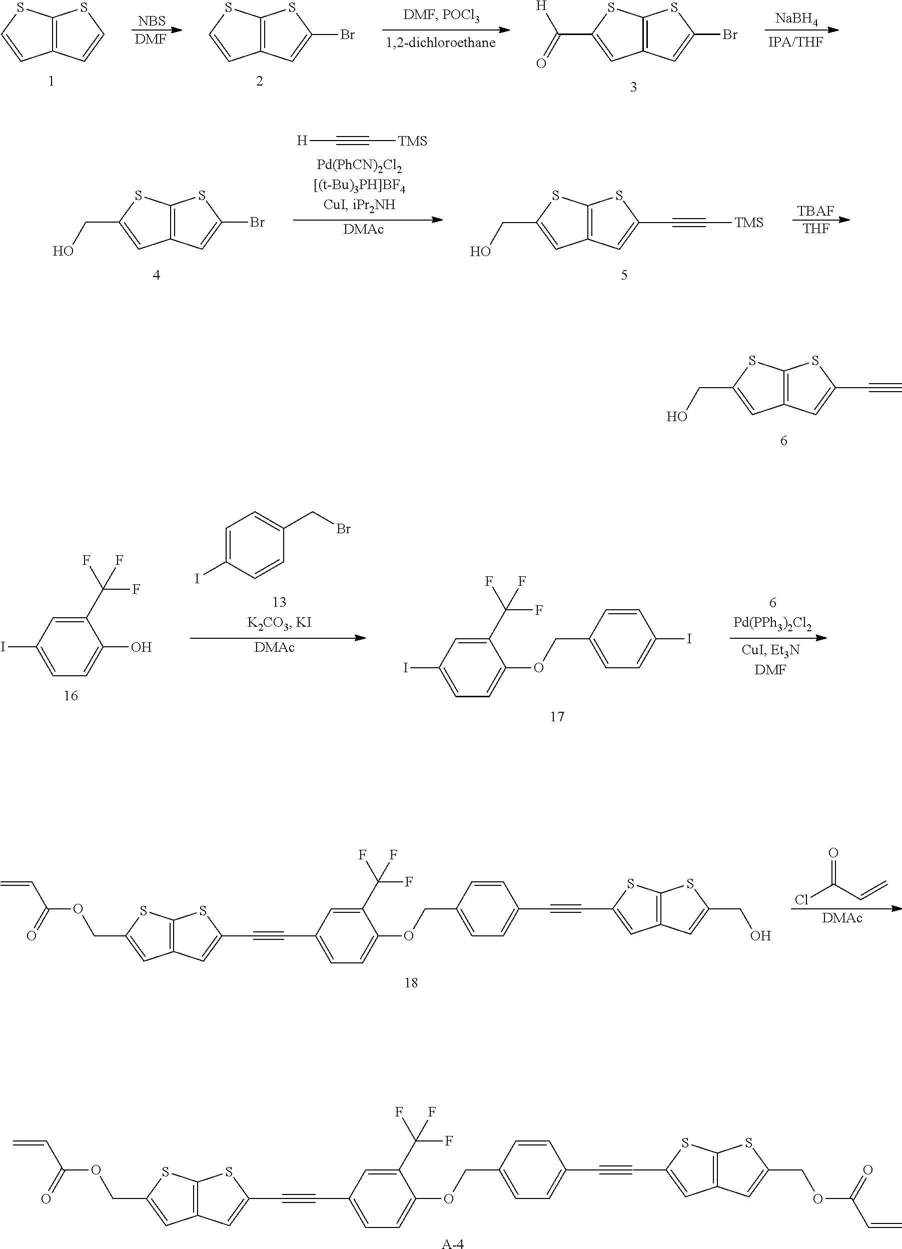

### (1) Synthesis of Compound 17

4-iodo-2-(trifluoromethyl) phenol (compound 16: 3.00 g, 10.4 mmol), 4-iodobenzyl bromide (compound 13: 3.17 g, 10.7 mmol), potassium carbonate (16.6 g, 12.0 mmol), and potassium iodide (0.17 g, 1.04 mmol) were dissolved in DMAc (15.0 mL), and the mixture was stirred at 45° C. for 30 minutes and then stirred at 55° C. for 3 hours. After temperature was decreased to room temperature, MeOH (5 mL) was added thereto, and the mixture was stirred for 10 minutes. Subsequently, a mixed solvent of water and MeOH (60 mL) was added dropwise thereto, and water (60 mL) was added dropwise thereto, and the mixture was stirred for 1 hour. The residue obtained by filtering the reaction solution was purified by flash column chromatography to obtain a compound 17 (2.01 g, yield: 38.3%).

### (2) Compound A-4

The ¹H-NMR of the obtained compound A-4 (CDCl₃) is shown below.

¹H-NMR of compound A-4 (CDCl₃): δ=5.23 (s, 2H), 5.38 (s, 4H), 5.88 (dd, 2H), 6.15 (dd, 2H), 6.47 (dd, 2H), 7.00 (d, 1H), 7.24 (s, 2H), 7.35 (d, 2H), 7.43 (d, 2H), 7.55 (d, 2H), 7.60 (dd, 1H), 7.78 (d, 1H)

### Synthesis Example 5: Synthesis of Compound A-5

The compound A-5 was synthesized according to the following scheme. The compound A-5 was synthesized by the same procedure as in (1) to (5) of <Synthesis Example 1: synthesis of compound A-1>, except that the compound 21 was used.

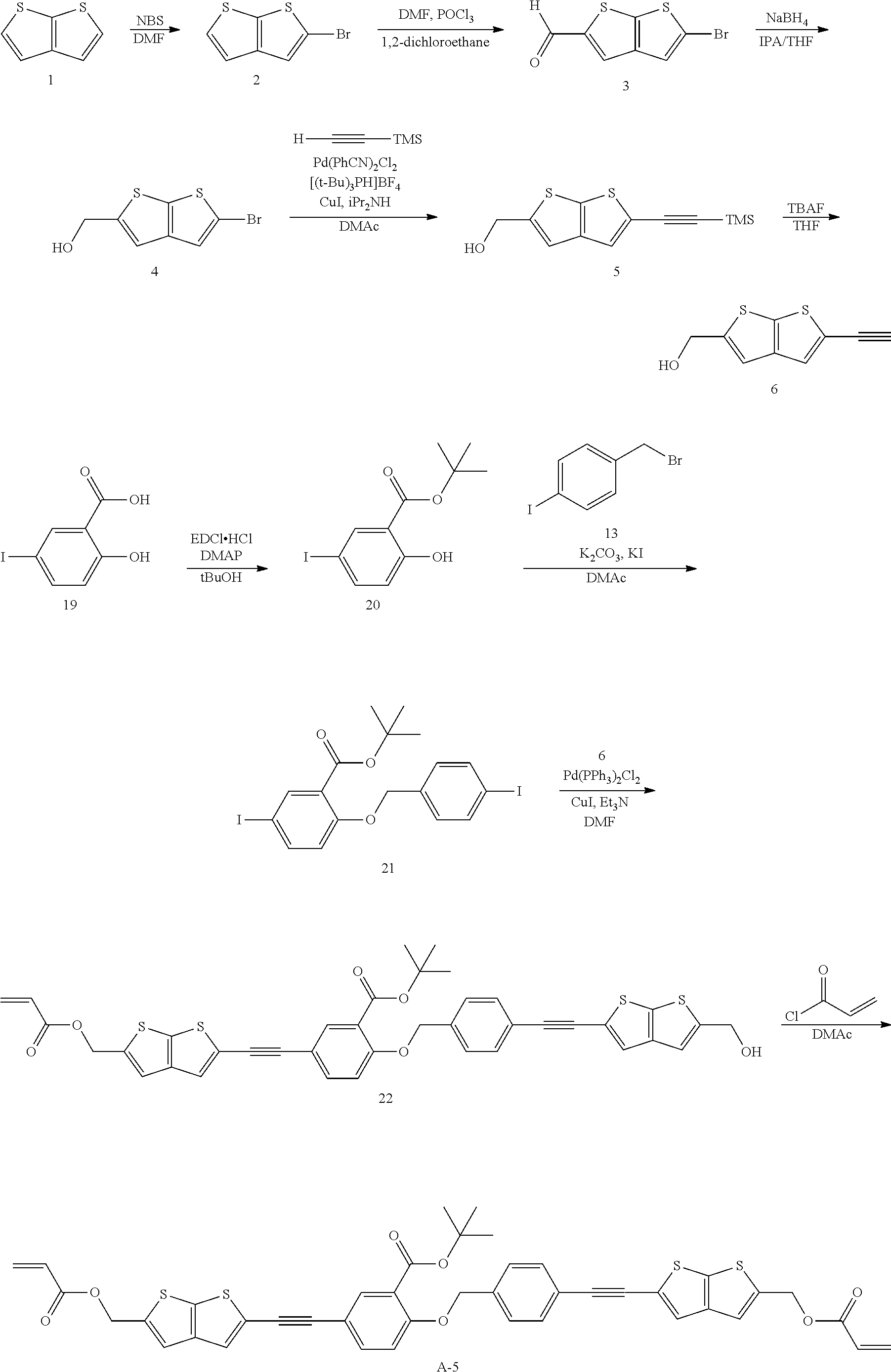

### (1) Synthesis of Compound 20

5-iodosalicylic acid (compound 19: 6.00 g, 22.8 mmol) and 4-(dimethylamino)pyridine (DMAP, 0.28 g, 2.27 mmol) were dissolved in t-butyl alcohol (60.0 mL), and a mixed solution of 1-ethyl-3-(3-dimethylaminopropyl) carbodiimide hydrochloride (EDIC·HCl, 6.53 g, 34.1 mmol) and THF (32.7 mL) was added dropwise thereto. After stirring at room temperature for 5 hours, the solvent was distilled off under reduced pressure, and the resulting residue was purified by flash column chromatography. A compound 20 (3.01 g, yield: 45.0%) was obtained.

### (2) Synthesis of Compound 21

The compound 20 (3.00 g, 9.37 mmol), 4-iodobenzyl bromide (compound 13: 2.78 g, 9.37 mmol), potassium carbonate (1.49 g, 10.8 mmol), and potassium iodide (0.16 g, 0.94 mmol) were dissolved in DMAc (15.0 mL), and the mixture was stirred at 45° C. for 30 minutes and then stirred at 55° C. for 3 hours. After temperature was decreased to room temperature, MeOH (5 mL) was added thereto, and the mixture was stirred for 10 minutes. Subsequently, a mixed solvent of water and MeOH (60 mL) was added dropwise thereto, and water (60 mL) was added dropwise thereto, and the mixture was stirred for 1 hour. The reaction solution was filtered to obtain a compound 21 (4.86 g, yield: 96.8%).

### (3) Compound A-5

The ¹H-NMR of the obtained compound A-5 (CDCl₃) is shown below.

¹H-NMR of compound A-5 (CDCl₃): δ=1.56 (s, 9H), 5.19 (s, 2H), 5.38 (d, 4H), 5.87 (dd, 2H), 6.13 (dd, 2H), 6.45 (dd, 2H), 6.95 (d, 1H), 7.23 (d, 2H), 7.34 (d, 2H), 7.47 (d, 2H), 7.52-7.56 (m, 3H), 7.87 (ds, 1H)

### Synthesis Example 6: Synthesis of Compound A-6

The compound A-6 was synthesized according to the following scheme. The compound 4 was synthesized by the same procedure as in (1) of <Synthesis Example 1: synthesis of compound A-1>.

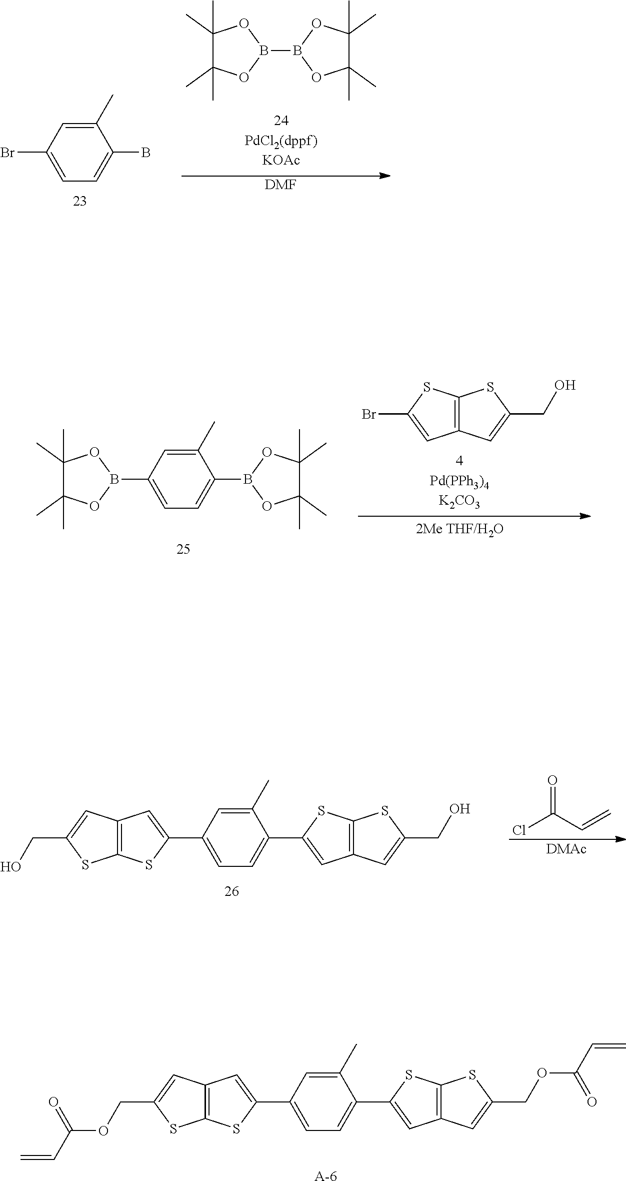

### (1) Synthesis of Compound 25

Under a nitrogen atmosphere, 2,5-dibromotoluene (compound 23: 10.0 g, 40.0 mmol), bis(pinacolato)diboron (compound 24: 25.6 g, 100.8 mmol), and potassium acetate (9.90 g, 100.8 mmol) were dissolved in anhydrous DMF, and then dichloro[1,1′-bis(diphenylphosphino) ferrocene]palladium (II) (2.93 g, 4.00 mmol) was added thereto, and the mixture was stirred at 90° C. for 12 hours. The solvent was distilled off under reduced pressure, and the resulting residue was purified by flash column chromatography, thereby obtaining a compound 25 (6.88 g, yield: 50.0%).

### (2) Synthesis of Compound 26

Under a nitrogen atmosphere, the compound 25 (3.0 g, 4.01 mmol), the compound 4 (2.50 g, 10.0 mmol), and potassium carbonate (4.16 g, 30.1 mmol) were dissolved in 2-methyl THF (75.0 mL) and water (30.0 mL), and then tetrakis(triphenylphosphine) palladium (46.4 mg, 0.40 mmol) was added thereto, and the mixture was stirred at 80° C. for 10 hours. After cooling to room temperature, the reaction solution was extracted with chloroform. The obtained organic layer was dried over magnesium sulfate, and the organic layer was filtered. The solvent was distilled off under reduced pressure, and the resulting residue was crystallized and purified with a mixed solvent of THF and MeOH to obtain a compound 26 (0.91 g, yield: 53.1%).

### (3) Synthesis of Compound A-6

The compound 26 (0.81 g, 1.88 mmol) was dissolved in DMAc (4.1 mL), acryloyl chloride (0.61 mL, 7.56 mmol) was added dropwise thereto, and the mixture was stirred at room temperature for 2 hours.

Methanol (8.1 mL) was added thereto, and the mixture was stirred at room temperature for 1 hour. Subsequently, the reaction solution was filtered, and the resulting residue was purified by flash column chromatography to obtain a compound A-6 (0.71 g, yield: 70.0%).

¹H-NMR of compound A-6 (CDCl₃): δ=2.50 (s, 3H), 5.39 (d, 4H), 5.88 (dd, 2H), 6.17 (dd, 2H), 6.45 (dd, 2H), 7.16 (s, 1H), 7.29 (s, 1H), 7.43-7.45 (m, 4H)

### Synthesis Example 7: Synthesis of Compound A-7

The compound A-7 was synthesized according to the following scheme. The compound A-7 was synthesized by the same procedure as in (1) to (3) of <Synthesis Example 6: synthesis of compound A-6>, except that the compound 27 (4,4′-diiodo-2,2′-dimethylbiphenyl) was used.

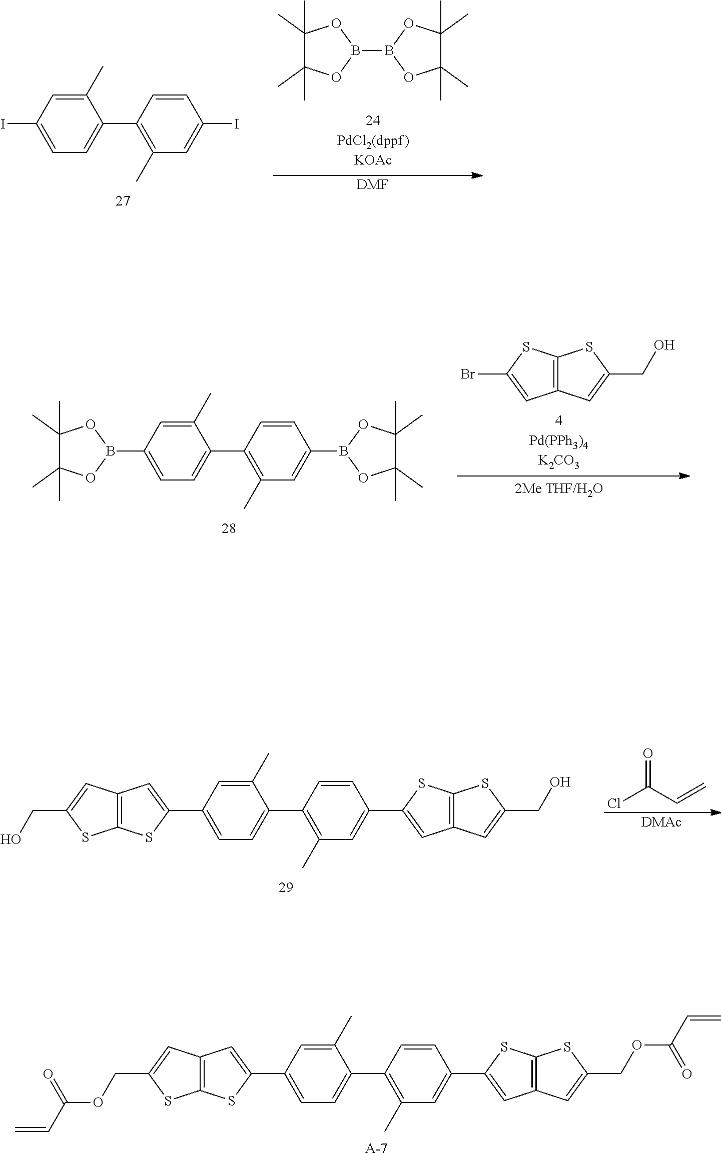

¹H-NMR of compound A-7 (CDCl₃): δ=2.16 (s, 6H), 5.40 (s, 4H), 5.88 (dd, 2H), 6.16 (dd, 2H), 6.48 (dd, 2H), 7.14 (d, 2H), 7.28 (s, 2H), 7.44 (s, 2H), 7.47-7.52 (m, 4H)

### Synthesis of Comparative Example Compound (Compound B-1)

According to WO2019/182129A, a compound B-1 was synthesized as a comparative compound.

### Specific Compound and Comparative Example Compound

The specific compounds (compound A-1 to A-7) and the comparative example compound (compound B-1) synthesized in the upper part are shown below.

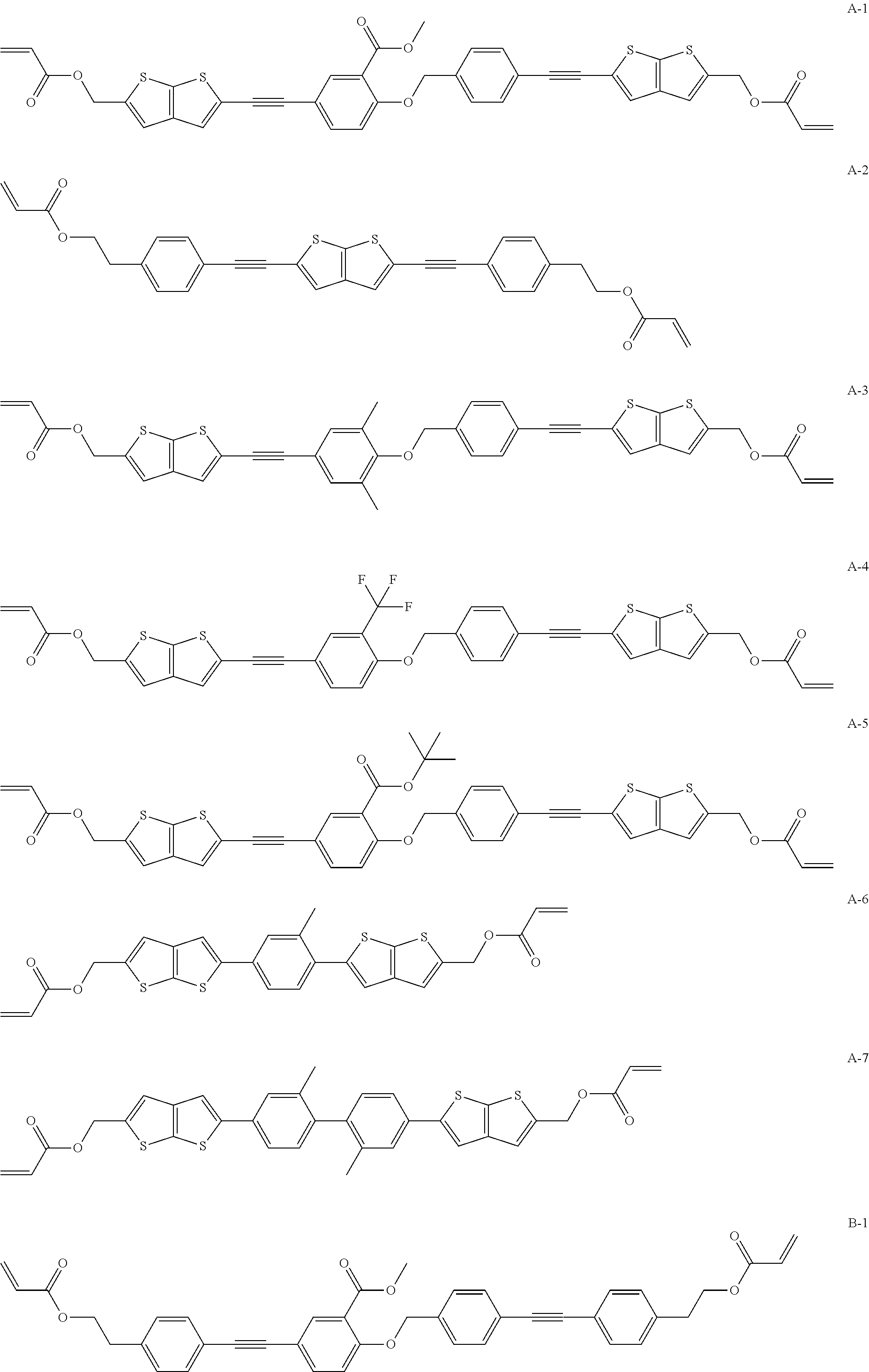

### Examples 1 to 7 and Comparative Example 1

Hereinafter, as Examples 1 to 7, the following evaluations were performed using the compounds A-1 to A-7 synthesized in the upper part. In addition, as Comparative Example 1, the following evaluations were performed using the compound B-1 synthesized in the upper part.

### [Evaluation]

### <Refractive Index and Birefringence Index>

The refractive index and the birefringence index of the optically anisotropic layer produced using the composition containing each of the compounds (compound A-1 to A-7 and compound B-1) of Examples and Comparative Example were evaluated.

### (Production of Optically Anisotropic Layer for Measuring Refractive Index and Birefringence Index)

First, a coating composition E having the following composition was prepared. The compound B-1 added together with each of the compounds of Examples and Comparative Example is the same as the compound B-1 synthesized in the upper part as a comparative compound. That is, in the coating composition E in Comparative Example 1, the total amount of the compound B-1 is 100 parts by mass.

Composition of coating composition E

| | |
|---|---|
| Each of compounds of Examples and Comparative | 50 parts by mass |
| Example shown in Table 1 | |
| Compound B-1 | 50 parts by mass |
| Polymerization initiator (manufactured by BASF | 3 parts by mass |
| SE, OMNIRAD (registered trademark) 819) | |
| The following leveling agent T-1 | 0.4 parts by mass |
| Chloroform | 1500 parts by mass |

The leveling agent T-1 is a compound having the following structure.

### —Leveling Agent T-1—

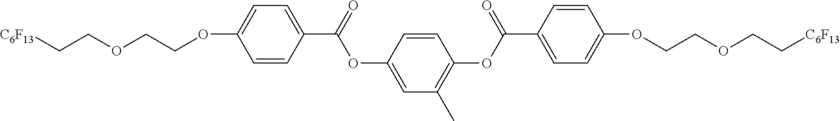

The coating composition E was spin-coated on the glass with the rubbed alignment film. The glass with the coating film of the obtained coating composition E was heated on a hot plate to a temperature at which the coating composition E exhibited a nematic phase, and then further irradiated with ultraviolet rays of 300 mJ/cm² through a filter that cut light having a wavelength of 350 nm or less on the hot plate to produce an optically anisotropic layer.

### (Evaluation of Refractive Index and Birefringence Index)

The refractive index of the produced optically anisotropic layer was measured using an ellipsometer (manufactured by J. A. Woollam Co., Ltd., M-2000). Specifically, the measurement was performed at incidence angles of 50°, 60°, and 70°, and the refractive index was calculated by performing Cauchy fitting using the measured values at 450 to 1700 nm. The refractive index ne for extraordinary rays and the birefringence index Δn (that is, the difference between ne and no) at a wavelength of 550 nm were calculated, and the results were classified according to the following evaluation standard.

The evaluation is preferably “B” or more and most preferably “A”. The results are shown in the columns of “ne” and “An” in Table 1.

The evaluation is preferably “B” or more and most preferably “A”.

### <<Evaluation Standard of Refractive Index Ne>>

$$“ A ” \text{:} 1.9 \leq ne .$$

$$“ B ” \text{:} 1.85 \leq ne < 1.9 .$$

$$“ C ” \text{:} ne < 1.85 .$$

<<Evaluation Standard of Birefringence Index Δn>>

$$“ A ” \text{:} 0.3 \leq Δ n .$$

$$“ B ” \text{:} 0.25 \leq Δ n < 0.3 .$$

$$“ C ” \text{:} Δ n < 0.25 .$$

### <Evaluation of Pattern Aligning Properties>

### (Formation of Alignment Film)

The following coating liquid for forming an alignment film was added dropwise onto the glass, and the glass was rotated at 500 rpm for 5 seconds and then rotated at 2,500 rpm for 20 seconds to perform the coating. The support on which the coating film of the coating liquid for forming an alignment film had been formed was dried using a hot plate at 60° C. for 60 seconds to form an alignment film.

Coating liquid for forming alignment film

| | |
|---|---|
| Material D for photo alignment | 4.00 part by mass |
| Water | 16.00 parts by mass |
| Butoxyethanol | 42.00 parts by mass |
| Propylene glycol monomethyl ether | 42.00 parts by mass |

The material D for photo alignment is a compound having the following structure.

### —Material D for Photo Alignment—

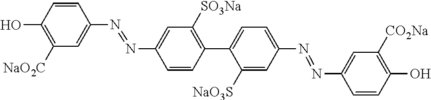

### (Exposure of Alignment Film)

The alignment film was exposed using the exposure device of FIG. 5 of WO2020/022496A to form an alignment film P-1 having an alignment pattern.

In the exposure device, a laser that emits a laser beam having a wavelength of 325 nm was used as the laser. The exposure amount of the interference light was 2,000 mJ/cm². It is noted that one period (the length over which the optical axis derived from the liquid crystal compound rotates) 180° of an alignment pattern formed by interference of two laser beams was controlled by changing the intersecting angle (the intersecting angle β) between the two beams.

### (Evaluation of Pattern Aligning Properties)

### <<Preparation of Composition for Measurement>>

As a composition for measuring the evaluation of pattern aligning properties, the following coating composition F was prepared. The compound B-1 added together with each of the compounds of Examples and Comparative Example is the same as the compound B-1 synthesized in the upper part as a comparative compound. That is, in the coating composition F in Comparative Example 1, the total amount of the compound B-1 is 100 parts by mass.

Coating composition F

| | |
|---|---|
| Each of compounds of Examples and | 50 parts by mass |
| Comparative Example shown in Table 1 | |
| Compound B-1 | 50 parts by mass |
| Chiral agent (manufactured by BASF SE, | 6 parts by mass |
| Paliocolor (registered trademark) LC756) | |
| Polymerization initiator (manufactured by | 2 parts by mass |
| BASF SE, OMNIRAD (registered trademark) 819) | |
| The leveling agent T-1 | 0.011 parts by mass |
| Chloroform | 972 parts by mass |

First, the coating composition F was added dropwise onto the alignment film P-1, and the coating composition F was rotated at 1,500 rpm for 10 seconds using a spin coater to perform the coating. Next, the obtained coating film was heated, cooled, and further cured by ultraviolet irradiation to produce a cured layer (liquid crystal immobilization layer).

The obtained cured layer was observed with a polarizing microscope to check the presence or absence of orientation defects, and the evaluation was performed according to the following evaluation standard. The results are shown in the column of “Pattern aligning properties” in Table 1.

The evaluation is preferably “B” or more and most preferably “A”.

### <<Evaluation Standard>>

“A”: No orientation defect.
“B”: Partial orientation defect is observed.
“C”: Orientation defect is observed over the entire surface.

Table 1 is shown below.

TABLE 1

| | | Evaluation result | | |
|---|---|---|---|---|
| | Compound | | | Pattern |
| | number | ne | Δn | alignment |
| Example 1 | A-1 | A | A | A |
| Example 2 | A-2 | A | A | B |
| Example 3 | A-3 | A | A | A |
| Example 4 | A-4 | A | A | A |
| Example 5 | A-5 | A | A | A |
| Example 6 | A-6 | A | A | B |
| Example 7 | A-7 | A | A | A |
| Comparative | B-1 | C | C | C |
| Example 1 | | | | |

From the results of Table 1, it is revealed that the optically anisotropic layer obtained by curing the composition containing the specific compound has a high refractive index ne for extraordinary rays and a high birefringence index Δn, and the specific compound has excellent pattern aligning properties.

In addition, from the comparison of Examples, it was confirmed that in a case where at least two selected from among n1 pieces of A¹ or one piece of A² in Formula (I) represented the fused-ring structure represented by Formula (Ia), the performance was excellent.

In addition, from the comparison of Examples, it was confirmed that in a case where n1 was an integer of 3 or more in Formula (I), the pattern aligning properties of the specific compound were more excellent.

In addition, it was confirmed that the optically anisotropic layer pattern-aligned and produced in Example 1 was optically adhered to the light guide plate, and the light extraction from the light guide plate was possible.

### EXPLANATION OF REFERENCES

1, 2, 3: optically anisotropic layer
xy plane: sheet surface
z direction: thickness direction
30: liquid crystal compound
Λ: length of single period
30A: optical axis derived from liquid crystal compound 30
θ: angle
R: region
d: thickness (film thickness) of optically anisotropic layer
P_{L}: levorotatory circularly polarized light
P_{R}: dextrorotatory circularly polarized light
L₁, L₄, L₆: incident ray
L₂, L₅, L₇: transmitted ray
Q1, Q2: absolute phase
E1, E2: equiphase surface
A₁, A₂, A₃: direction

What is claimed is:

1. A compound represented by Formula (I), 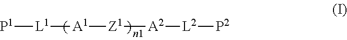
in Formula (I),
P¹ and P² each independently represent a hydrogen atom, a polymerizable group, or a monovalent substituent, where at least one of P¹ or P² is a polymerizable group,
L¹ and L² each independently represent a single bond or an alkylene group having 20 or less carbon atoms, where any —CH₂— of the alkylene group may be substituted with —O—, —S—, —NR—, —CO—, or —CS—, any —(CH₂)₂— of the alkylene group may be substituted with —CH═CH— or —C≡C—, and any hydrogen atom of the alkylene group may be substituted with a fluorine atom or a chlorine atom,
Z¹ represents a single bond, —O—, —S—, —CHRCHR—, —OCHR—, —CO—, —SO—, —SO₂—, —COO—, —CO—S—, —O—CO—O—, —CO—NR—, —SCHR—, —SO—CHR—, —SO₂—CHR—, —CF₂O—, —CF₂S—, —OCHRCHRO—, —SCHRCHRS—, —SO—CHRCHR—SO—, —SO₂—CHRCHR—SO₂—, —CH═CH—COO—, —CH—CH—OCO—, —CH—CH—CONR—, —CH—CH—COS—, —COO—CHRCHR—, —OCO—CHRCHR—, —COO—CHR—, —OCO—CHR—, —CR—CR—, —CR≡N—, —N≡CR—, —N≡N—, —CR═N—N═CR—, —CF═CF—, —C≡C—C≡C—, —C≡C—, or an alkylene group having 10 or less carbon atoms, where any —CH₂— of the alkylene group may be substituted with —O—, —S—, —NR—, —CO—, or —CS—, any —(CH₂)₂— of the alkylene group may be substituted with —CH═CH— or —C≡C—, and any hydrogen atom of the alkylene group may be substituted with a fluorine atom or a chlorine atom, provided that in a case where a plurality of Z¹'s are present in the formula, the plurality of Z¹'s may be the same as or different from each other,
R represents a hydrogen atom or an alkyl group having 1 to 10 carbon atoms,
n represents an integer of 1 to 7, and
A¹ and A² each independently represent a divalent aromatic ring group which may have a substituent or a divalent alicyclic group which may have a substituent, where at least one selected from among n1 pieces of A¹ and one piece of A² in the formula is a fused-ring structure represented by Formula (Ia), provided that in a case where a plurality of A¹'s are present in the formula, the plurality of A¹'s may be the same as or different from each other, 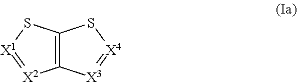
in Formula (Ia), two of X¹, X², X³, and X⁴ represent —C(*)═, and the other two of X¹, X², X³, and X⁴ each independently represent —N═ or —C(R^{A})—, R^{A} represents a hydrogen atom or a substituent, and * represents a bonding position.

2. The compound according to claim 1,
wherein, in Formula (Ia), two of X¹, X², X³, and X⁴ represent —C(*)—, and the other two of X¹, X², X³, and X⁴ represent —C(R^{A})═.

3. The compound according to claim 1,
wherein, in Formula (Ia), X¹ and X⁴ represent —C(*)═.

4. The compound according to claim 1,
wherein, at least one selected from among n1 pieces of Z¹'s present in Formula (I) is —C≡C—.

5. The compound according to claim 1,
wherein, in Formula (I), at least two selected from among n1 pieces of A¹ or one piece of A² are each the fused-ring structure represented by Formula (Ia).

6. The compound according to claim 1,
wherein, in Formula (I), at least two selected from among n1 pieces of A¹ or one piece of A² are each the fused-ring structure represented by Formula (Ia), and X¹ and X⁴ in the fused-ring structure represented by Formula (Ia) represent —C(*)═.

7. The compound according to claim 1,
wherein, in Formula (I), at least one of P¹ or P² represents a polymerizable group selected from the group consisting of Formula (P-1) to Formula (P-19),

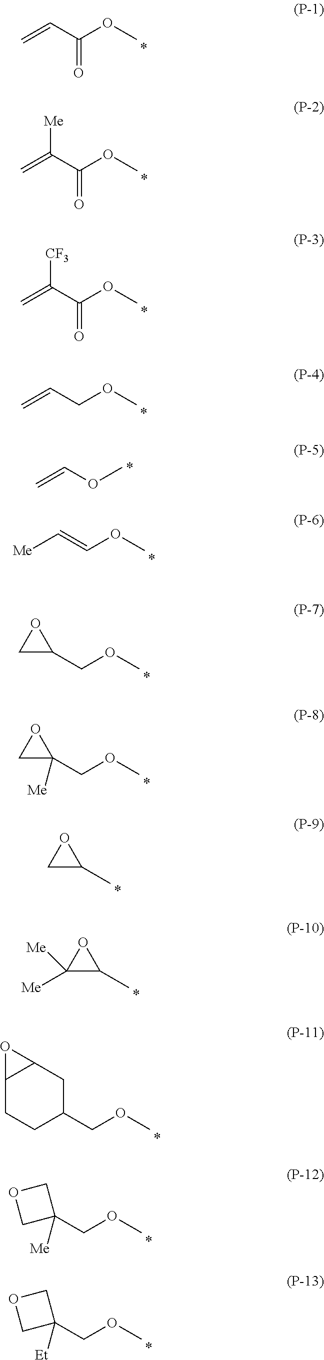

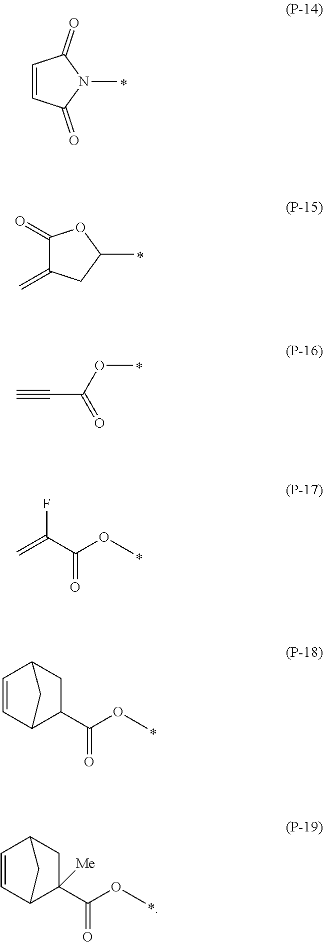

8. The compound according to claim 7,
wherein, in Formula (I), the at least one of P¹ or P² represents a polymerizable group selected from Formula (P-1) or Formula (P-2).

9. The compound according to claim 1,
wherein, in Formula (I), n1 represents an integer of 3 to 7.

10. The compound according to claim 1,
wherein A¹ and A² each independently represent a divalent aromatic hydrocarbon ring group which may have a substituent, a divalent aromatic heterocyclic ring group which may have a substituent and is constituted of an aromatic heterocyclic ring having 5 to 6 membered rings, a fused-ring represented by Formula (Ia), or a divalent alicyclic ring which may have a substituent, where at least one selected from among n1 pieces of A¹ and one piece of A² in the formula is the fused-ring structure represented by Formula (Ia).

11. A composition comprising:
the compound according to claim 1.

12. The composition according to claim 11, further comprising:
a polymerization initiator.

13. The composition according to claim 12, further comprising:
a chiral agent.

14. The composition according to claim 13,
wherein the composition has liquid crystallinity.

15. A cured product that is obtained by curing the composition according to claim 11.

16. An optically anisotropic body that is obtained by curing the composition according to claim 11.

17. The optically anisotropic body according to claim 16,
wherein the optically anisotropic body has an alignment pattern in which an orientation of an optical axis derived from the compound continuously rotates in at least one in-plane direction.

18. An optical element comprising:
the optically anisotropic body according to claim 17.

19. A light guide element comprising:
the optical element according to claim 18; and
a light guide plate.

20. The compound according to claim 2,
wherein, in Formula (Ia), X¹ and X⁴ represent —C(*)═.
